SEQUENCE LISTING (74 sequences)

SEQ ID NO: 1 - RNA, 77 nt, synthetic construct
  source 1..77: mol_type = other RNA
ccggcggtagttcagcagggcagaacggcggactctaaatccgcatggcgccggttcaaatccggcccgccggacca

SEQ ID NO: 2 - RNA, 76 nt, synthetic construct
  source 1..76: mol_type = other RNA
ccggcggtagttcagagggcagaacggcggactctaaatccgcatggcgccagttcaaatctggcccgccggacca

SEQ ID NO: 3 - RNA, 76 nt, synthetic construct
  source 1..76: mol_type = other RNA
cccgccttagttcagagggcagaacggcggactctaaatccgcatggcgccggttcaaatccggcaggcgggacca

SEQ ID NO: 4 - RNA, 76 nt, synthetic construct
  source 1..76: mol_type = other RNA
ccggcggtagttcagagggcagaacggcggactctaaatccgcatggcgccggttcaaatccggcccgccggacca

SEQ ID NO: 5 - RNA, 76 nt, synthetic construct
  source 1..76: mol_type = other RNA
cccgccttagttcagagggcagaacggcggactctaaatccgcatggcacgggttcaaatcccgtaggcgggacca

SEQ ID NO: 6 - RNA, 76 nt, synthetic construct
  source 1..76: mol_type = other RNA
ccggcggtagttcagagggcagaacggcggactctaaatccgcatggcaggggttcaaatcccctccgccggacca

SEQ ID NO: 7 - RNA, 77 nt, synthetic construct
  source 1..77: mol_type = other RNA
cccgccttagttcagcagggcagaacggcggactctaaatccgcaggtcgccggttcaaatccggcaggcgggacca

SEQ ID NO: 8 - RNA, 76 nt, synthetic construct
  source 1..76: mol_type = other RNA
cccgccttagttcagagggcagaacggcggactctaaatccgcaggtcgccggttcaaatccggcaggcgggacca

SEQ ID NO: 9 - RNA, 77 nt, synthetic construct
  source 1..77: mol_type = other RNA
cccgccttagttcagcagggcagaacggcggactctaaatccgcaggtcacgggttcaaatcccgtaggcgggacca

SEQ ID NO: 10 - RNA, 76 nt, synthetic construct
  source 1..76: mol_type = other RNA
ccggcggtagttcagagggcagaacggcggactctaaatccgcatggcacgggttcaaatcccgtccgccggacca

SEQ ID NO: 11 - RNA, 76 nt, synthetic construct
  source 1..76: mol_type = other RNA
ccggcggtagttcagagggcagaacggcggactctaaatccgcaggtcaggggttcaaatcccctccgccggacca

SEQ ID NO: 12 - RNA, 77 nt, synthetic construct
  source 1..77: mol_type = other RNA
ccggcggtagttcagcagggcagaacggcggactctaaatccgcatggcaggggttcaaatcccctccgccggacca

SEQ ID NO: 13 - RNA, 76 nt, synthetic construct
  source 1..76: mol_type = other RNA
ccggcggtagttcagagggcagaacggcggactctaaatccgcaggtcacgggttcaaatcccgtccgccggacca

SEQ ID NO: 14 - RNA, 77 nt, synthetic construct
  source 1..77: mol_type = other RNA
ccggaggtagttcagcagggcagaacggcggactctaaatccgcatggcgccggttcaaatccggcccttcggacca

SEQ ID NO: 15 - RNA, 76 nt, synthetic construct
  source 1..76: mol_type = other RNA
ccggtgttagttcagagggcagaacggcggactctaaatccgcatgtcacgggttcaaatcccgtacaccggacca

SEQ ID NO: 16 - RNA, 76 nt, synthetic construct
  source 1..76: mol_type = other RNA
ccgaccttagttcagagggcagaacggcggactctaaatccgcatgtcaggggttcaaatcccctaggtcggacca

SEQ ID NO: 17 - DNA, 76 bp, synthetic construct
  source 1..76: mol_type = other DNA
ccggcggtagttcagagggcagaacggcggactctaaatccgcatggcgccagttcaaatctggcccgccggacca

SEQ ID NO: 18 - DNA, 76 bp, synthetic construct
  source 1..76: mol_type = other DNA
cccgccttagttcagagggcagaacggcggactctaaatccgcatggcgccggttcaaatccggcaggcgggacca

SEQ ID NO: 19 - DNA, 76 bp, synthetic construct
  source 1..76: mol_type = other DNA
ccggcggtagttcagagggcagaacggcggactctaaatccgcatggcgccggttcaaatccggcccgccggacca

SEQ ID NO: 20 - DNA, 76 bp, synthetic construct
  source 1..76: mol_type = other DNA
cccgccttagttcagagggcagaacggcggactctaaatccgcatggcacgggttcaaatcccgtaggcgggacca

SEQ ID NO: 21 - DNA, 76 bp, synthetic construct
  source 1..76: mol_type = other DNA
ccggcggtagttcagagggcagaacggcggactctaaatccgcatggcaggggttcaaatcccctccgccggacca

SEQ ID NO: 22 - DNA, 77 bp, synthetic construct
  source 1..77: mol_type = other DNA
cccgccttagttcagcagggcagaacggcggactctaaatccgcaggtcgccggttcaaatccggcaggcgggacca

SEQ ID NO: 23 - DNA, 76 bp, synthetic construct
  source 1..76: mol_type = other DNA
cccgccttagttcagagggcagaacggcggactctaaatccgcaggtcgccggttcaaatccggcaggcgggacca

SEQ ID NO: 24 - DNA, 77 bp, synthetic construct
  source 1..77: mol_type = other DNA
cccgccttagttcagcagggcagaacggcggactctaaatccgcaggtcacgggttcaaatcccgtaggcgggacca

SEQ ID NO: 25 - DNA, 76 bp, synthetic construct
  source 1..76: mol_type = other DNA
ccggcggtagttcagagggcagaacggcggactctaaatccgcatggcacgggttcaaatcccgtccgccggacca

SEQ ID NO: 26 - DNA, 76 bp, synthetic construct
  source 1..76: mol_type = other DNA
ccggcggtagttcagagggcagaacggcggactctaaatccgcaggtcaggggttcaaatcccctccgccggacca

SEQ ID NO: 27 - DNA, 77 bp, synthetic construct
  source 1..77: mol_type = other DNA
ccggcggtagttcagcagggcagaacggcggactctaaatccgcatggcaggggttcaaatcccctccgccggacca

SEQ ID NO: 28 - DNA, 76 bp, synthetic construct
  source 1..76: mol_type = other DNA
ccggcggtagttcagagggcagaacggcggactctaaatccgcaggtcacgggttcaaatcccgtccgccggacca

SEQ ID NO: 29 - DNA, 77 bp, synthetic construct
  source 1..77: mol_type = other DNA
ccggaggtagttcagcagggcagaacggcggactctaaatccgcatggcgccggttcaaatccggcccttcggacca

SEQ ID NO: 30 - DNA, 76 bp, synthetic construct
  source 1..76: mol_type = other DNA
ccggtgttagttcagagggcagaacggcggactctaaatccgcatgtcacgggttcaaatcccgtacaccggacca

SEQ ID NO: 31 - DNA, 76 bp, synthetic construct
  source 1..76: mol_type = other DNA
ccgaccttagttcagagggcagaacggcggactctaaatccgcatgtcaggggttcaaatcccctaggtcggacca

SEQ ID NO: 32 - RNA, 77 nt, Methanocaldococcus jannaschii
  source 1..77: mol_type = genomic RNA
ccggcggtagttcagcctggtagaacggcggactgtagatccgcatgtcgctggttcaaatccggcccgccggacca

SEQ ID NO: 33 - DNA, 77 bp, Methanocaldococcus jannaschii
  source 1..77: mol_type = genomic DNA
ccggcggtagttcagcctggtagaacggcggactgtagatccgcatgtcgctggttcaaatccggcccgccggacca

SEQ ID NO: 34 - DNA, 77 bp, synthetic construct
  source 1..77: mol_type = other DNA
  misc_difference 2: note = a, c, t, g, unknown, or other
  misc_difference 3: note = a, c, t, g, unknown, or other
  misc_difference 6: note = a, c, t, g, unknown, or other
  misc_difference 7: note = a, c, t, g, unknown, or other
  misc_difference 51: note = a, c, t, g, unknown, or other
  misc_difference 65: note = a, c, t, g, unknown, or other
  misc_difference 67: note = a, c, t, g, unknown, or other
  misc_difference 68: note = a, c, t, g, unknown, or other
  misc_difference 71: note = a, c, t, g, unknown, or other
  misc_difference 72: note = a, c, t, g, unknown, or other
cnngcnntagttcagcagggcagaacggcggactctaaatccgcatggcwnrggttcaaatccynwnngcnngacca

SEQ ID NO: 35 - protein, 306 aa, Methanocaldococcus jannaschii
MDEFEMIKRNTSEIISEEELREVLKKDEKSAVIGFEPSGKIHLGHYLQIKKMIDLQNAGFDIIIYLADLHAYLNQKGELDEIRKIGDYNKKVFEAMGLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 36 - RNA, 76 nt, synthetic construct
  source 1..76: mol_type = other RNA
  misc_difference 16: note = May be absent
ccsrhsktagttcagcagggcagaacggcggactctaaatccgcakgkcrsnrgtcaaatcynsymsdysggacca

SEQ ID NO: 37 - RNA, 77 nt, synthetic construct
  source 1..77: mol_type = other RNA
ccggcggtagttcagcagggcagaacggcggactctaaatccgcatggcgctggttcaaatccagcccgccggacca

SEQ ID NO: 38 - RNA, 77 nt, synthetic construct
  source 1..77: mol_type = other RNA
ccggcggtagttcagcagggcagaacggcggactctaaatccgcatggcgcaggttcaaatcctgcccgccggacca

SEQ ID NO: 39 - protein, 306 aa, synthetic construct
MDEFEMIKRNTSEIVSEEELREVLKKDEKSAVIGFEPSGKIHLGHYLQIKKMIDLQNAGFDIIIYLADLHAYLNQKGELDEIRKIGDYNKKVFEAMGLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 40 - protein, 306 aa, synthetic construct
MDEFEMIKRNASEIVSEEELREVLKKDEKSAVIGFEPSGKIHLGHYLQIKKMIDLQNAGFDIIIYLADLHAYLNQKGELDEIRKIGDYNKKVFEAMGLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 41 - protein, 306 aa, synthetic construct
MDEFEMIKRNTSEIVSEEELREVLKKGEKSAVIGFEPSGKIHLGHYLQIKKMIDLQNAGFDIIIYLADLHAYLNQKGELDEIRKIGDYNKKVFEAMGLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 42 - protein, 306 aa, synthetic construct
MDEFEMIKRNTSEIVSEEELREVLKKDEKSAVIGFEPSGKIHLGHYLQIKKMIDLQNAGFDIIIYLADLHAYLNQKGELDEIRKIGDYNKKVFEAIGLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 43 - protein, 306 aa, synthetic construct
MDEFEMIKRNTSEIVSEEELREVLKKDEKSAVIGFEPSGKIHLGHYLQIKKMIDLQNAGFDIIIYLADLHAYLNQKGELDEIRKIGDYNKKVFEAMDLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 44 - protein, 306 aa, synthetic construct
MDEFEMIKRNTSEIVSEEELREVLKKDEKSAVIGFEPSGKIHLGHYLQIKKMIDLQNAGFDIIIYLADLHAYLNQKGELDEIRKIGDYNKKVFEAMGLKARYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 45 - protein, 306 aa, Methanocaldococcus jannaschii
MDEFEMIKRNTSEIISEEELREVLKKDEKSAVIGFEPSGKIHLGHYLQIKKMIDLQNAGFDIIIYLADLHAYLNQKGELDEIRKIGDYNKKVFEAMGLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 46 - protein, 306 aa, synthetic construct
MDEFEMIKRNASEIVSEEELREVLKKGEKSAVIGFEPSGKIHLGHYLQIKKMIDLQNAGFDIIIYLADLHAYLNQKGELDEIRKIGDYNKKVFEAIDLKARYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 47 - protein, 306 aa, synthetic construct
MDEFEMIKRNASEIVSEEELREVLKKDEKSAVIGFEPSGKIHLGHYLQIKKMIDLQNAGFDIIIYLADLHAYLNQKGELDEIRKIGDYNKKVFEAIGLKARYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 48 - protein, 306 aa, synthetic construct
MDEFEMIKRNASEIVSEEELREVLKKGEKSAVIGFEPSGKIHLGHYLQIKKMIDLQNAGFDIIIYLADLHAYLNQKGELDEIRKIGDYNKKVFEAIDLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 49 - protein, 306 aa, synthetic construct
MDEFEMIKRNASEIVSEEELREVLKKDEKSAVIGFEPSGKIHLGHYLQIKKMIDLQNAGFDIIIYLADLHAYLNQKGELDEIRKIGDYNKKVFEAMDLKARYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 50 - protein, 306 aa, synthetic construct
MDEFEMIKRNASEIVSEEELREVLKKGEKSAVIGFEPSGKIHLGHYLQIKKMIDLQNAGFDIIIYLADLHAYLNQKGELDEIRKIGDYNKKVFEAIGLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 51 - protein, 306 aa, synthetic construct
MDEFEMIKRNASEIVSEEELREVLKKGEKSAVIGFEPSGKIHLGHYLQIKKMIDLQNAGFDIIIYLADLHAYLNQKGELDEIRKIGDYNKKVFEAIDLKARYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKPGGDLTVNSYEELESLFKNKELHSMRLKNAVAEELIKILEPIRKRL

SEQ ID NO: 52 - protein, 306 aa, synthetic construct
MDEFEMIKRNASEIVSEEELREVLKKGEKSAVIGFEPSGKIHLGHYLQIKKMIDLQNAGFDIIIYLADLHAYLNQKGELDEIRKIGDYNKKVFEAIDLKARYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKWPEKPGGDLTVNSYEELESLFKNKELHPMRLKNAVAEELIKILEPIRKRL

SEQ ID NO: 53 - protein, 306 aa, synthetic construct
MDEFEMIKRNASEIVSEEELREVLKKGEKSAVIGFEPSGKIHLGHYLQIKKMIDLQNAGFDIIIYLADLHAYLNQKGELDEIRKIGDYNKKVFEAIDLKARYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKWPEKPGGDLTVNSYEELESLFKNKELHSMRLKNAVAEELIKILEPIRKRL

SEQ ID NO: 54 - protein, 306 aa, synthetic construct
MDEFEMIKRNASEIVSEEELREVLKKGEKSAVIGFEPSGKIHLGHYLQIKKMIDLQNAGFDIIIYLADLHAYLNQKGELDEIRKIGDYNKKVFEAIDLKARYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKWPEKPGGDLTVNSYEELESLFKNKELHPMYLKNAVAEELIKILEPIRKRL

SEQ ID NO: 55 - protein, 306 aa, synthetic construct
MDEFEMIKRNASEIVSEEELREVLKKGEKSAVIGFEPSGKIHLGHYLQIKKMIDLQNAGFDIIIYLADLHAYLNQKGELDEIRKIGDYNKKVFEAIDLKARYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMYLKNAVAEELIKILEPIRKRL

SEQ ID NO: 56 - protein, 306 aa, synthetic construct
MDEFEMIKRNASEIVSEEELREVLKKGEKSAVIGFEPSGKIHLGHYLQIKKMIDLQNAGFDIIIYLADLHAYLNQKGELDEIRKIGDYNKKVFEAIDLKARYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKPGGDLTVNSYEELESLFKNKELHPMYLKNAVAEELIKILEPIRKRL

SEQ ID NO: 57 - DNA, 921 bp, synthetic construct
  source 1..921: mol_type = other DNA
atggatgagtttgagatgattaaacgtaatacgagtgaaattgtcagtgaggaagaattgcgcgaggtccttaaaaaagatgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccatgggtcttaaagcgaaatatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagtttggcggggatttaacagttaacagctatgaagaacttgaaagtcttttcaaaaataaggagttgcatccgatgcgcttaaagaacgctgtcgcggaggaattgattaaaatcctggagcctattcgtaaacgtctgtaa

SEQ ID NO: 58 - DNA, 921 bp, synthetic construct
  source 1..921: mol_type = other DNA
atggatgagtttgagatgattaaacgtaatgcgagtgaaattgtcagtgaggaagaattgcgcgaggtccttaaaaaaggcgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccattgatcttaaagcgcgttatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagtttggcggggatttaacagttaacagctatgaagaacttgaaagtcttttcaaaaataaggagttgcatccgatgcgcttaaagaacgctgtcgcggaggaattgattaaaatcctggagcctattcgtaaacgtctgtaa

SEQ ID NO: 59 - DNA, 921 bp, synthetic construct
  source 1..921: mol_type = other DNA
atggatgagtttgagatgattaaacgtaatgcgagtgaaattgtcagtgaggaagaattgcgcgaggtccttaaaaaagatgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccatgggtcttaaagcgaaatatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagtttggcggggatttaacagttaacagctatgaagaacttgaaagtcttttcaaaaataaggagttgcatccgatgcgcttaaagaacgctgtcgcggaggaattgattaaaatcctggagcctattcgtaaacgtctgtaa

SEQ ID NO: 60 - DNA, 921 bp, synthetic construct
  source 1..921: mol_type = other DNA
atggatgagtttgagatgattaaacgtaatacgagtgaaattgtcagtgaggaagaattgcgcgaggtccttaaaaaaggcgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccatgggtcttaaagcgaaatatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagtttggcggggatttaacagttaacagctatgaagaacttgaaagtcttttcaaaaataaggagttgcatccgatgcgcttaaagaacgctgtcgcggaggaattgattaaaatcctggagcctattcgtaaacgtctgtaa

SEQ ID NO: 61 - DNA, 921 bp, synthetic construct
  source 1..921: mol_type = other DNA
atggatgagtttgagatgattaaacgtaatacgagtgaaattgtcagtgaggaagaattgcgcgaggtccttaaaaaagatgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccattggtcttaaagcgaaatatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagtttggcggggatttaacagttaacagctatgaagaacttgaaagtcttttcaaaaataaggagttgcatccgatgcgcttaaagaacgctgtcgcggaggaattgattaaaatcctggagcctattcgtaaacgtctgtaa

SEQ ID NO: 62 - DNA, 921 bp, synthetic construct
  source 1..921: mol_type = other DNA
atggatgagtttgagatgattaaacgtaatacgagtgaaattgtcagtgaggaagaattgcgcgaggtccttaaaaaagatgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccatggatcttaaagcgaaatatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagtttggcggggatttaacagttaacagctatgaagaacttgaaagtcttttcaaaaataaggagttgcatccgatgcgcttaaagaacgctgtcgcggaggaattgattaaaatcctggagcctattcgtaaacgtctgtaa

SEQ ID NO: 63 - DNA, 921 bp, synthetic construct
  source 1..921: mol_type = other DNA
atggatgagtttgagatgattaaacgtaatacgagtgaaattgtcagtgaggaagaattgcgcgaggtccttaaaaaagatgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccatgggtcttaaagcgcgttatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagtttggcggggatttaacagttaacagctatgaagaacttgaaagtcttttcaaaaataaggagttgcatccgatgcgcttaaagaacgctgtcgcggaggaattgattaaaatcctggagcctattcgtaaacgtctgtaa

SEQ ID NO: 64 - DNA, 921 bp, synthetic construct
  source 1..921: mol_type = other DNA
atggatgagtttgagatgattaaacgtaatgcgagtgaaattgtcagtgaggaagaattgcgcgaggtccttaaaaaagatgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccattggtcttaaagcgcgttatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagtttggcggggatttaacagttaacagctatgaagaacttgaaagtcttttcaaaaataaggagttgcatccgatgcgcttaaagaacgctgtcgcggaggaattgattaaaatcctggagcctattcgtaaacgtctgtaa

SEQ ID NO: 65 - DNA, 921 bp, synthetic construct
  source 1..921: mol_type = other DNA
atggatgagtttgagatgattaaacgtaatgcgagtgaaattgtcagtgaggaagaattgcgcgaggtccttaaaaaaggcgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccattgatcttaaagcgaaatatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagtttggcggggatttaacagttaacagctatgaagaacttgaaagtcttttcaaaaataaggagttgcatccgatgcgcttaaagaacgctgtcgcggaggaattgattaaaatcctggagcctattcgtaaacgtctgtaa

SEQ ID NO: 66 - DNA, 921 bp, synthetic construct
  source 1..921: mol_type = other DNA
atggatgagtttgagatgattaaacgtaatgcgagtgaaattgtcagtgaggaagaattgcgcgaggtccttaaaaaagatgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccatggatcttaaagcgcgttatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagtttggcggggatttaacagttaacagctatgaagaacttgaaagtcttttcaaaaataaggagttgcatccgatgcgcttaaagaacgctgtcgcggaggaattgattaaaatcctggagcctattcgtaaacgtctgtaa

SEQ ID NO: 67 - DNA, 921 bp, synthetic construct
  source 1..921: mol_type = other DNA
atggatgagtttgagatgattaaacgtaatgcgagtgaaattgtcagtgaggaagaattgcgcgaggtccttaaaaaaggcgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccattggtcttaaagcgaaatatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagtttggcggggatttaacagttaacagctatgaagaacttgaaagtcttttcaaaaataaggagttgcatccgatgcgcttaaagaacgctgtcgcggaggaattgattaaaatcctggagcctattcgtaaacgtctgtaa

SEQ ID NO: 68 - DNA, 921 bp, synthetic construct
  source 1..921: mol_type = other DNA
atggatgagtttgagatgattaaacgtaatgcgagtgaaattgtcagtgaggaagaattgcgcgaggtccttaaaaaaggcgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccattgatcttaaagcgcgttatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagccgggcggggatttaacagttaacagctatgaagaacttgaaagtcttttcaaaaataaggagttgcatagcatgcgcttaaagaacgctgtcgcggaggaattgattaaaatcctggagcctattcgtaaacgtctgtaa

SEQ ID NO: 69 - DNA, 921 bp, synthetic construct
  source 1..921: mol_type = other DNA
atggatgagtttgagatgattaaacgtaatgcgagtgaaattgtcagtgaggaagaattgcgcgaggtccttaaaaaaggcgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccattgatcttaaagcgcgttatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaatggcccgagaagccgggcggggatttaacagttaacagctatgaagaacttgaaagtcttttcaaaaataaggagttgcatccgatgcgcttaaagaacgctgtcgcggaggaattgattaaaatcctggagcctattcgtaaacgtctgtaa

SEQ ID NO: 70 - DNA, 921 bp, synthetic construct
  source 1..921: mol_type = other DNA
atggatgagtttgagatgattaaacgtaatgcgagtgaaattgtcagtgaggaagaattgcgcgaggtccttaaaaaaggcgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccattgatcttaaagcgcgttatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaatggcccgagaagccgggcggggatttaacagttaacagctatgaagaacttgaaagtcttttcaaaaataaggagttgcatagcatgcgcttaaagaacgctgtcgcggaggaattgattaaaatcctggagcctattcgtaaacgtctgtaa

SEQ ID NO: 71 - DNA, 921 bp, synthetic construct
  source 1..921: mol_type = other DNA
atggatgagtttgagatgattaaacgtaatgcgagtgaaattgtcagtgaggaagaattgcgcgaggtccttaaaaaaggcgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccattgatcttaaagcgcgttatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaatggcccgagaagccgggcggggatttaacagttaacagctatgaagaacttgaaagtcttttcaaaaataaggagttgcatccgatgtatttaaagaacgctgtcgcggaggaattgattaaaatcctggagcctattcgtaaacgtctgtaa

SEQ ID NO: 72 - DNA, 921 bp, synthetic construct
  source 1..921: mol_type = other DNA
atggatgagtttgagatgattaaacgtaatgcgagtgaaattgtcagtgaggaagaattgcgcgaggtccttaaaaaaggcgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccattgatcttaaagcgcgttatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagtttggcggggatttaacagttaacagctatgaagaacttgaaagtcttttcaaaaataaggagttgcatccgatgtatttaaagaacgctgtcgcggaggaattgattaaaatcctggagcctattcgtaaacgtctgtaa

SEQ ID NO: 73 - DNA, 921 bp, synthetic construct
  source 1..921: mol_type = other DNA
atggatgagtttgagatgattaaacgtaatgcgagtgaaattgtcagtgaggaagaattgcgcgaggtccttaaaaaaggcgaaaaatcggccgttatcggcttcgagccaagtggtaaaatccatttagggcactacctgcagatcaaaaagatgattgatttacagaacgcgggctttgacatcattatctaccttgctgacttgcatgcgtatttgaaccaaaagggcgagcttgatgaaatccgcaaaattggagattacaataaaaaagttttcgaagccattgatcttaaagcgcgttatgtctacgggtctgaacacggtttggataaggattacactttgaacgtatatcgcttggcgcttaaaaccactttgaaacgtgctcgccgctctatggaattaattgcacgcgaggacgaaaaccctaaagtcgcggaggtcatctaccctatcatgcaggtaaacggtatccattatgagggggttgatgtagctgttggtggtatggagcaacgtaaaattcacatgctggcacgcgagcttctgccgaaaaaggttgtctgtattcacaacccggttcttacggggttagatggagagggaaaaatgagctctagcaagggcaatttcattgcggtagatgactctccggaagaaattcgtgcaaaaatcaagaaggcgtactgtccggccggcgtagtcgaaggaaatccgattatggagattgcaaagtactttctggagtatccattgacgatcaaacgccccgagaagccgggcggggatttaacagttaacagctatgaagaacttgaaagtcttttcaaaaataaggagttgcatccgatgtatttaaagaacgctgtcgcggaggaattgattaaaatcctggagcctattcgtaaacgtctgtaa

SEQ ID NO: 74 - RNA, 10 nt, synthetic construct
  source 1..10: mol_type = other RNA
cagagggcag

(19) United States
(12) Patent Application Publication
(10) Pub. No.: US 2023/0407363 A1
(43) Pub. Date: Dec. 21, 2023

(54) tRNA SYNTHETASES AND METHODS OF USE THEREOF

(71) Applicant: Absci Corporation, Vancouver, WA (US)

(72) Inventor: Yi Zhang, Vancouver, WA (US)

(21) Appl. No.: 18/132,176

(22) Filed: Apr. 7, 2023

Related U.S. Application Data
(60) Provisional application No. 63/328,854, filed on Apr. 8, 2022.

(51) Int. Cl. C12P 21/02; C12N 15/70; C12N 9/00

(52) U.S. Cl. CPC C12P 21/02; C12N 15/70; C12N 9/93; C12Y 601/01001; C12N 2800/101

(57) ABSTRACT

This disclosure provides engineered aminoacyl tRNA synthetases and tRNAs for efficient production of proteins containing non-standard amino acids. These engineered orthogonal tRNA (O-tRNA)/orthogonal aminoacyl tRNA synthetase (O-RS) pairs, i.e., Orthogonal Translation Systems (OTSs) can be used to incorporate a non-standard amino acid in a specific position in a growing polypeptide in response to a selector codon that is recognized by the engineered tRNA.

### CROSS REFERENCE TO RELATED APPLICATIONS

This application claims the benefit of U.S. Provisional Application No. 63/328,854 filed Apr. 8, 2022, which is hereby incorporated in its entirety by reference for all purposes.

### SEQUENCE LISTING

The instant application contains a Sequence Listing which has been submitted electronically in XML format and is hereby incorporated by reference in its entirety. Said XML copy, created on Jul. 27, 2023, is named ABS-020US_SL.xml and is 93,257 bytes in size.

### BACKGROUND

Although proteins are ubiquitously involved in almost all biological processes, the vast majority of them are biosynthesized from only 20 standard amino acids with a limited set of functional groups (amines, carboxylic acids, amides, alcohols, thiols, etc.). One goal of synthetic biology is to expand the chemical alphabets utilized by living organisms. The ability to incorporate non-standard amino acids (nsAAs) into proteins is a highly desirable feature for a protein expression system as it enables novel chemistry which streamlines the production of previously difficult-to-produce molecules. For example, incorporation of nsAAs into biologics that support site-specific conjugation under mild aqueous conditions would provide a highly simplified route for the attachment of half-life extension moieties (e.g., PEG), uniform glycan structures, and antibody-drug conjugates (ADCs).

The incorporation of nsAAs into proteins and peptides is typically achieved by the expression of an engineered transfer RNA (tRNA)/aminoacyl tRNA synthetase pair, i.e., Orthogonal Translation Systems (OTSs), that can aminoacylate the nsAA of interest onto the novel tRNA. To achieve efficient production of the desired protein containing nsAA, the engineered tRNA must be selectively aminoacylated by its cognate aminoacyl-tRNA synthetase (aaRS) while remaining inactive to all endogenous aaRSs in the protein expression system. The resulting aminoacyl-tRNA must be efficiently recognized by elongation factor Tu (EF-Tu) to be translocated to the A site of ribosome; after binding to the ribosomal A site, the aminoacyl-tRNA must function efficiently in translation as a substrate for peptidyl transferase; and finally the tRNA bearing the growing peptide chain must be translocated to the P site, undergo another acyl transfer reaction, and be released from the ribosome.

The performance of an OTS can be defined in terms of two main parameters, fidelity and efficiency. Fidelity refers to the accuracy with which the reassigned codon is read through by the ribosome as the nsAA versus misread as any other amino acid. A lower fidelity OTS will result in a mixture of proteins harboring nsAA and one or more other amino acids at each reassigned codon, which significantly complicates the purification process or even renders the separation impossible. Efficiency reflects how effectively the ribosome is able to read through the reassigned codon in the presence of the OTS and the nsAA. A less efficient nsAA-incorporation system will result in a lower yield of the protein of interest. A desirable OTS should have a balanced profile in terms of both fidelity and efficiency during the entire course of fermentation. Ideally, the mischarging of standard amino acid is negligible while the read through efficiency is close to if not better than translation of the wild type DNA sequence.

OTSs of various sources have been engineered and used for production of proteins containing nsAA in bacterial protein expression systems. For example, OTSs from archaea Methanococcus jannaschii were engineered for a number of new amino acids with novel chemical, physical or biological properties, including photoaffinity labels and photoisomerizable amino acids, photocrosslinking amino acids (see Chin, J. W., et al. (2002) Proc. Natl. Acad. Sci. U.S.A. 99:11020-11024; and, Chin, J. W., et al., (2002) J. Am. Chem. Soc. 124:9026-9027), keto amino acids (see, Wang, L., et al., (2003) Proc. Natl. Acad. Sci. U.S.A. 100:56-61 and Zhang, Z. et al., Biochem. 42(22):6735-6746 (2003)), heavy atom containing amino acids, and glycosylated amino acids have been incorporated efficiently and with high fidelity into proteins in E. coli in response to the amber codon (TAG). Several other orthogonal pairs have been reported, including: glutaminyl systems (see, e.g., Liu, D. R., and Schultz, P. G. (1999) Proc. Natl. Acad. Sci. U.S.A. 96:4780-4785), aspartyl systems (see, e.g., Pasternak, M., et al., (2000) Helv. Chim. Acta 83:2277 2286), tyrosyl systems (see, e.g., Ohno, S., et al., (1998). J. Bio Chem. (Tokyo, Jpn.) 124:1065-1068; and Kowal, A. K., et al., (2001) Proc. Natl. Acad. Sci. U.S.A. 98:2268-2273), and systems derived from S. cerevisiae tRNAs and synthetases have been described for the potential incorporation of non-standard amino acids in E. coli.

Although non-standard amino acids are typically incorporated into proteins with acceptable efficiency and fidelity, further systemic optimization for improved protein yields is highly desirable. The extent of incorporation of the non-standard amino acid varies-only in rare cases can be quantitative, which results in suboptimal quantity and quality profile of the product. Prior known OTSs showcased either reduced fidelity or efficiency during high cell-density fermentation of bacterial expression system. These defects are particularly pronounced in highly optimized bacterial expression systems which are dedicated to produce biologics. Therefore, to further expand the application scope of the nsAA, there is a need to develop improved and/or additional components of the OTSs, e.g., tRNA synthetases.

### SUMMARY

The present disclosure provides, in part, compositions for augmenting the protein biosynthetic machinery of a cell or cell-free translation system to accommodate additional genetically encoded amino acids using orthogonal tRNA (O-tRNA), an orthogonal aminoacyl tRNA synthetase (O-RS), and a non-standard amino acid, where the O-RS aminoacylates the O-tRNA with the selected amino acid. The O-tRNA recognizes a first selector codon and has suppression activity in the presence of a cognate synthetase in response to a selector codon. The cell uses the components to incorporate the selected amino acid into a growing polypeptide chain. A nucleic acid comprising a polynucleotide that encodes a polypeptide of interest can also be present, where the polynucleotide comprises a selector codon that is recognized by the O-tRNA.

In one aspect, the disclosure relates to an orthogonal tRNA synthetase (O-RS) comprising a substitution of at least one of the following residues as compared to a wild-type M. jannaschii tRNA synthetase (SEQ ID NO:45): T11, I15, D27, M96, G97, and K101. In certain embodiments, the orthogonal tRNA synthetase comprises at least 85% sequence identity to SEQ ID NO: 45 but is not identical to SEQ ID NO: 35. In certain embodiments, the O-RS comprises at least one of the following substitutions:
(a) T11A, T11V, T11I, T11L, or T11G;
(b) I15V, I15A, I15L, or I15G; (c) D27G, D27A, D27V, D27I, or D27L;
(c) M96I, M96A, M96V, M96L, or M96G;
(d) G97D or G97E; and
(e) K101R, or K101H.

In certain embodiments, the O-RS comprises at least one of the following substitutions: T11A, I15V, D27G, M96I, G97D, and K101R. In certain embodiments, the O-RS comprises an additional substitution of at least one of the following residues as compared to a wild-type M. jannaschii tRNA synthetase (SEQ ID NO:45): R257, F261, P284, M285, D286, and G158.

In certain embodiments, the O-RS comprises at least one of the following substitutions:
(a) R257W, R257F, R257Y, or R257H,
(b) F261P,
(c) P284S, P284A, P284G, P284C, or P284V,
(d) M285D, or M285E,
(e) D286Y, D286W, D286F, D286H, or D286R, and
(f) G158D, or G158E.

In certain embodiments, the O-RS comprises at least one of the following substitutions: R257W, F261P, P284S, M285D, D286Y, and G158D.

In certain embodiments, the O-RS comprises a substitution at residue D286. In certain embodiments, the O-RS comprises substitutions at residues 115 and D286. In certain embodiments, the substitution at D286 is a D286F, D286W, D286H, D286K, D286V, D286R, or a D286Y substitution. In certain embodiments, the substitution at D286 is D286Y. In certain embodiments, the O-RS comprises at least one of the substitutions I15V and D286R. In certain embodiments, the O-RS comprises the substitutions I15V and D286R.

In certain embodiments, the O-RS comprises an amino acid sequence that is at least 85% identical to a sequence selected from SEQ ID NOs: 39-44 and 46-56.

In certain embodiments, the amino acid sequence is at least 90% identical, at least 91% identical, at least 92% identical, at least 93% identical, at least 94% identical, at least 95% identical, at least 96% identical, at least 97% identical, at least 98% identical, or at least 98.7% identical to a sequence selected from SEQ ID NOs: 39-44 and 46-56. In certain embodiments, the amino acid sequence is selected from SEQ ID NOs: 39-44 and 46-56.

In another aspect, the disclosure relates to an orthogonal translation system (OTS) comprising the O-RS as described in the disclosure and an orthogonal tRNA (O-tRNA).

In certain embodiments, the O-tRNA comprises a nucleic acid sequence at least 85% identical to the sequence set forth in SEQ ID NO: 1 and comprising a deletion of the cytosine located at nucleic acid position 16 of the O-tRNA, wherein the nucleic acid positions correspond to the sequence set forth in SEQ ID NO: 1; and wherein the O-tRNA is capable of being aminoacylated with at least one non-standard amino acid (nsAA) by an orthogonal aminoacyl tRNA synthetase (O-RS).

In certain embodiments, the nucleic acid sequence is at least 90% identical, at least 91% identical, at least 92% identical, at least 93% identical, at least 94% identical, at least 95% identical, at least 96% identical, at least 97% identical, at least 98% identical, or at least 98.7% identical to the sequence set forth in SEQ ID NO: 1.

In certain embodiments, the O-tRNA comprises an adenine at nucleic acid position 53 and a uracil at nucleic acid position 63, wherein the nucleic acid positions correspond to the sequence set forth in SEQ ID NO:1. In certain embodiments, the O-tRNA comprises a nucleic acid sequence consisting of the sequence set forth in SEQ ID NO: 2.

In certain embodiments, the O-tRNA comprises a cytosine at nucleic acid positions 3 and 6; a uracil at nucleic acid position 7, an adenosine at nucleic acid position 67, and a guanine at nucleic acid positions 68 and 71, wherein the nucleic acid positions correspond to the sequence set forth in SEQ ID NO: 1.

In certain embodiments, the O-tRNA comprises a nucleic acid sequence consisting of the sequence set forth in SEQ ID NO: 3. In certain embodiments, the O-tRNA comprises a nucleic acid sequence consisting of the sequence set forth in SEQ ID NO: 4.

In certain embodiments, the O-tRNA comprises the sequence CAG-AGGGCAG (SEQ ID NO: 74) at nucleic acid positions 13 to 23, wherein the nucleic acid positions correspond to the sequence set forth in SEQ ID NO: 1.

In certain embodiments, the O-tRNA comprises a nucleic acid sequence consisting of a sequence set forth in SEQ ID NO: 36, wherein the sequence does not comprise SEQ ID NO: 1, SEQ ID NO: 37, or SEQ ID NO: 38.

In certain embodiments, the O-tRNA comprises a cytosine at position 3. In certain embodiments, the O-tRNA comprises an adenine at position 4. In certain embodiments, the O-tRNA comprises a uracil at position 5. In certain embodiments, the O-tRNA comprises a cytosine at position 6. In certain embodiments, the O-tRNA comprises a uracil at position 7.

In certain embodiments, the O-tRNA comprises a guanine at position 46. In certain embodiments, the O-tRNA comprises a uracil at position 48. In certain embodiments, the O-tRNA comprises an adenine at position 50. In certain embodiments, the O-tRNA comprises a guanine at position 51. In certain embodiments, the O-tRNA comprises an adenine at position 53. In certain embodiments, the O-tRNA comprises a uracil at position 63. In certain embodiments, the O-tRNA comprises a cytosine at position 65. In certain embodiments, the O-tRNA comprises a uracil at position 66. In certain embodiments, the O-tRNA comprises an adenine at position 67. In certain embodiments, the O-tRNA comprises a guanine at position 68. In certain embodiments, the O-tRNA comprises an adenine or a uracil at position 69. In certain embodiments, the O-tRNA comprises a uracil at position 70. In certain embodiments, the O-tRNA comprises a guanine at position 71. In certain embodiments, the O-tRNA comprises a cytosine at position 3, a cytosine at position 6, and a uracil at position 7. In certain embodiments, the O-tRNA comprises a guanine at position 46 and a uracil at position 48. In certain embodiments, the O-tRNA comprises an adenine at position 67, a guanine at position 68, and a guanine at position 71.

In certain embodiments, the O-tRNA comprises a nucleic acid sequence consisting of a sequence set forth in SEQ ID NO. 2-16.

In certain embodiments, the OTS further comprises a non-standard amino acid (nsAA).

In certain embodiments, the nsAA has the structure according to Formula I; wherein the R group is any substituent other than a corresponding substituent used in the twenty natural amino acids. In certain embodiments, the nsAA has the structure according to Formula I wherein the R group comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, or combinations thereof.

In certain embodiments, the nsAA is selected from the group consisting of: an amino acid comprising a photoactivatable cross-linker, a spin-labeled amino acid, a fluorescent amino acid, a metal binding amino acid, a metal containing amino acid, a radioactive amino acid, an amino acid comprising at least one novel functional group, an amino acid that covalently or noncovalently interacts with other molecules, a photocaged amino acid, a photoisomerizable amino acid, an amino acids comprising biotin or a biotin analogue, a carbohydrate-modified amino acid, an amino acid comprising polyethylene glycol or polyether, a heavy atom substituted amino acid, a chemically cleavable amino acid, a photocleavable amino acid, and combinations thereof.

In certain embodiments, the nsAA comprises a tyrosine analog. In certain embodiments, the tyrosine analog is selected from the group consisting of a para-substituted tyrosine, an ortho-substituted tyrosine, and a meta-substituted tyrosine. In certain embodiments, the substituted tyrosine comprises a keto group, an acetyl group, a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or combinations thereof.

In certain embodiments, the nsAA comprises a glutamine analog. In certain embodiments, the glutamine analog comprises an α-hydroxy derivative, a γ-substituted derivative, a cyclic derivative, or an amide substituted glutamine derivative.

In certain embodiments, the nsAA comprises a phenylalanine analog. In certain embodiments, the phenylalanine analog is an amino-, an isopropyl-, or an O-allyl-containing phenylalanine analog. In certain embodiments, the phenylalanine analog is selected from the group consisting of a para-substituted phenylalanine, an ortho-substituted phenylalanine, and a meta-substituted phenylalanine. In certain embodiments, the substituent comprises a hydroxy group, a methoxy group, a methyl group, an allyl group, an aldehyde, an azido, an iodo, a bromo, a keto group or an acetyl group.

In certain embodiments, the nsAA comprises a para-acetyl phenylalanine. In certain embodiments, the nsAA is selected from the group consisting of a p-propargyl phenylalanine, an O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, an isopropyl-L-phenylalanine, a 4-acetyl-phenylalanine (AcF), a 4-azido-phenylalanine (AzF); a 4-propargyloxyphenylalanine (PaF); a 4-aminophenylalanine (AmF), and a 4-azidomethyl-L-phenylalanine (mAzF). In certain embodiments, the nsAA comprises an O-methyl-L-tyrosine. In certain embodiments, the nsAA comprises an L-3-(2-naphthyl)alanine.

In certain embodiments, the O-tRNA recognizes a selector codon. In certain embodiments, the selector codon is an amber codon. In certain embodiments, the OTS further comprises a polynucleotide comprising at least one selector codon that is recognized by the O-tRNA. In certain embodiments, the OTS further comprises a mutant EF-Tu. In certain embodiments, the OTS is a cell-free translation system. In certain embodiments, the cell-free translation system is a cell lysate. In certain embodiments, the cell-free translation system is a reconstituted system. In certain embodiments, the OTS is a cellular translation system.

In another aspect, the disclosure relates to a cell comprising the OTS as described herein. In certain embodiments, the cell is a non-eukaryotic cell or a prokaryotic cell. In certain embodiments, the prokaryotic cell is Escherichia coli. In certain embodiments, the cell is a eukaryotic cell. In certain embodiments, the cell is a yeast cell. In certain embodiments, the cell is a fungal cell. In certain embodiments, the cell is a mammalian cell. In certain embodiments, the cell is an insect cell. In certain embodiments, the cell is a plant cell. In certain embodiments, the cell encodes a mutation in an EF-Tu. In certain embodiments, the cell has reduced expression of Release Factor 1 compared to an otherwise identical wild-type cell.

In another aspect, the disclosure relates to a polypeptide comprising at least one nsAA, wherein the polypeptide is produced by an OTS or a cell as described herein. In certain embodiments, the polypeptide comprises an antibody or antigen binding fragment thereof. In certain embodiments, the polypeptide comprises human growth hormone. In another aspect, the disclosure relates to a polynucleotide comprising a nucleic acid sequence encoding an O-RS as described herein. In another aspect, the disclosure relates to a polynucleotide comprising a nucleic acid sequence encoding an O-RS comprising a nucleic acid sequence consisting of a sequence set forth in any one of SEQ ID NOs. 57-73. In certain embodiments, the polynucleotide further comprises a nucleic acid sequence complementary to the O-RS sequence consisting of a sequence set forth in any one of SEQ ID NOs. 57-73.

In another aspect, the disclosure relates to a polynucleotide or set of polynucleotides comprising a nucleic acid sequence of an O-tRNA and a nucleic acid sequence encoding an O-RS as described herein. In certain embodiments, the O-tRNA comprises a nucleic acid sequence consisting of the sequence set forth in SEQ ID NO. 1.

In another aspect, the disclosure relates to a vector comprising at least one polynucleotide as described herein. In certain embodiments, the vector is an expression vector. In certain embodiments, the vector is selected from the group consisting of a plasmid, a cosmid, a phage, and a virus.

In another aspect, the disclosure relates to a cell comprising a polynucleotide as described herein or a vector as described herein.

In another aspect, the disclosure relates to a kit comprising one or more of the polynucleotide(s) described herein, one or more of the vectors describe herein, or one or more of the cells described herein and instructions for use.

In another aspect, the disclosure relates to a method of producing a polypeptide comprising at least one nsAA, comprising expressing in a cell an O-tRNA and an O-RS as described herein. In certain embodiments, the O-RS aminoacylates the O-tRNA with the nsAA.

In certain embodiments, the nsAA has the structure according to Formula I; and wherein the R group is any substituent other than a corresponding substituent used in the twenty natural amino acids.

In certain embodiments, the nsAA has the structure according to Formula I; and wherein the R group comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, or combinations thereof.

In certain embodiments, the nsAA is selected from the group consisting of an amino acid comprising a photoactivatable cross-linker, a spin-labeled amino acid, a fluorescent amino acid, a metal binding amino acid, a metal containing amino acid, a radioactive amino acid, an amino acid with at least one novel functional group, an amino acid that covalently or noncovalently interacts with other molecules, a photocaged amino acid, a photoisomerizable amino acid, an amino acid comprising biotin or a biotin analogue, a carbohydrate-modified amino acid, and an amino acid comprising polyethylene glycol or polyether, a heavy atom substituted amino acid, a chemically cleavable amino acid, a photocleavable amino acid, and combinations thereof.

In certain embodiments, the nsAA comprises a tyrosine analog. In certain embodiments, the tyrosine analog is selected from the group consisting of a para-substituted tyrosine, an ortho-substituted tyrosine, and a meta-substituted tyrosine. In certain embodiments, the substituted tyrosine comprises a keto group, an acetyl group, a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or combinations thereof.

In certain embodiments, the nsAA comprises a glutamine analog. In certain embodiments, the glutamine analog comprises a α-hydroxy derivative, a γ-substituted derivative, a cyclic derivative, or an amide substituted glutamine derivative.

In certain embodiments, the nsAA comprises a phenylalanine analog. In certain embodiments, the phenylalanine analog is an amino-, an isopropyl-, or a 0-allyl-containing phenylalanine analog. In certain embodiments, the phenylalanine analog is selected from the group consisting of a para-substituted phenylalanine, an ortho-substituted phenylalanine, and a meta-substituted phenylalanine. In certain embodiments, the substituent comprises a hydroxy group, a methoxy group, a methyl group, an allyl group, an aldehyde, an azido, an iodo, a bromo, a keto group or an acetyl group.

In certain embodiments, the nsAA comprises a para-acetyl phenylalanine. In certain embodiments, the nsAA is selected from the group consisting of a p-propargyl phenylalanine, a O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, and an isopropyl-L-phenylalanine.

In certain embodiments, the nsAA is selected from the group consisting of 4-acetyl-phenylalanine (AcF), 4-azido-phenylalanine (AzF); 4-propargyloxyphenylalanine (PaF), 4-aminophenylalanine (AmF) and 4-azidomethyl-phenylalanine (mAzF). In certain embodiments, the nsAA comprises a 4-acetyl-phenylalanine (AcF). In certain embodiments, the nsAA comprises a 4-azido-phenylalanine (AzF) or 4-azidomethyl-phenylalanine (mAzF). In certain embodiments, the nsAA comprises a 4-propargyloxyphenylalanine (PaF). In certain embodiments, the nsAA comprises a 4-aminophenylalanine (AmF).

In certain embodiments, the nsAA is biosynthesized by the cell. In certain embodiments, the nsAA is provided to the cell exogenously. In certain embodiments, the cell is a non-eukaryotic cell or a prokaryotic cell. In certain embodiments, the prokaryotic cell is Escherichia coli. In certain embodiments, the cell is a eukaryotic cell. In certain embodiments, the eukaryotic cell is a yeast cell. In certain embodiments, the eukaryotic cell is a fungal cell. In certain embodiments, the eukaryotic cell is a mammalian cell. In certain embodiments, the eukaryotic cell is an insect cell. In certain embodiments, the eukaryotic cell is a plant cell.

In certain embodiments, the O-tRNA recognizes a selector codon. In certain embodiments, the selector codon is an amber codon.

In certain embodiments, the polypeptide comprises an antibody or antigen binding fragment thereof. In certain embodiments, the polypeptide comprises human growth hormone.

In another aspect, the disclosure relates to a method of producing a polypeptide comprising at least one non-standard amino acid (nsAA), comprising providing:
i) an O-tRNA, wherein the O-tRNA is capable of being aminoacylated with at least one non-standard amino acid (nsAA) by an O-RS as described herein;
ii) the O-RS; wherein the O-RS aminoacylates the O-tRNA with the nsAA; and
iii) a polynucleotide encoding the polypeptide, wherein the polynucleotide comprises at least one selector codon; and wherein the O-tRNA recognizes the selector codon.

In certain embodiments, the nsAA has the structure according to Formula I; and wherein the R group is any substituent other than a corresponding substituent used in the twenty natural amino acids.

In certain embodiments, the nsAA has the structure according to Formula I; and wherein the R group comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, or combinations thereof.

In certain embodiments, the nsAA is selected from the group consisting of an amino acid comprising a photoactivatable cross-linker, a spin-labeled amino acid, a fluorescent amino acid, a metal binding amino acid, a metal containing amino acid, a radioactive amino acid, an amino acid with at least one novel functional group, an amino acid that covalently or noncovalently interacts with other molecules, a photocaged amino acid, a photoisomerizable amino acid, an amino acid comprising biotin or a biotin analogue, a carbohydrate-modified amino acid, an amino acid comprising polyethylene glycol or polyether, a heavy atom substituted amino acid, a chemically cleavable amino acid, a photocleavable amino acid, and combinations thereof.

In certain embodiments, the nsAA comprises a tyrosine analog. In certain embodiments, the tyrosine analog is selected from the group consisting of a para-substituted tyrosine, an ortho-substituted tyrosine, and a meta-substituted tyrosine. In certain embodiments, the substituted tyrosine comprises a keto group, an acetyl group, a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or combinations thereof.

In certain embodiments, the nsAA comprises a glutamine analog. In certain embodiments, the glutamine analog comprises a α-hydroxy derivative, a γ-substituted derivative, a cyclic derivative, an amide substituted glutamine derivative.

In certain embodiments, the nsAA comprises a phenylalanine analog. In certain embodiments, the phenylalanine analog is an amino-, an isopropyl-, or an O-allyl-containing phenylalanine analog. In certain embodiments, the phenylalanine analog is selected from the group consisting of a para-substituted phenylalanine, an ortho-substituted phenylalanine, and a meta-substituted phenylalanine. In certain embodiments, the substituent comprises a hydroxy group, a methoxy group, a methyl group, an allyl group, an aldehyde, an azido, an iodo, a bromo, a keto group or an acetyl group.

In certain embodiments, the nsAA comprises a para-acetyl phenylalanine. In certain embodiments, the nsAA is selected from the group consisting of a p-propargyl phenylalanine, an O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, and an isopropyl-L-phenylalanine.

In certain embodiments, the nsAA is selected from the group consisting of 4-acetyl-phenylalanine (AcF), 4-azido-phenylalanine (AzF); 4-propargyloxyphenylalanine (PaF), 4-aminophenylalanine (AmF), and 4-azidomethyl-phenylalanine (mAzF). In certain embodiments, the nsAA comprises a 4-acetyl-phenylalanine (AcF). In certain embodiments, the nsAA comprises a 4-azido-phenylalanine (AzF) or 4-azidomethyl-phenylalanine (mAzF). In certain embodiments, the nsAA comprises a 4-propargyloxyphenylalanine (PaF). In certain embodiments, the nsAA comprises a 4-aminophenylalanine (AmF).

In certain embodiments, the selector codon is an amber codon. In certain embodiments, the polypeptide comprises an antibody or antigen binding fragment thereof. In certain embodiments, the polypeptide comprises human growth hormone. In certain embodiments, the polypeptide is produced by a cell-free translation system. In certain embodiments, the cell-free translation system is a cell lysate. In certain embodiments, the cell-free translation system is a reconstituted system.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is an illustration of a plasmid map for the MP6 mutagenic plasmid used for the directed evolution of O-RS sequences.

FIG. 2A and FIG. 2B provide illustrations of two exemplary assay plasmids for the directed evolution of O-RS sequences.

FIG. 3 is an illustration of a plasmid map for exemplary constructs containing candidate OTSs according to the disclosure.

FIG. 4 is an illustration of a plasmid map for the assay construct containing the GFP reporter for evaluation of the incorporation efficiency of an OTS.

FIGS. 5A-5G provide graphs showing average RRE, MMF and OD scores for an OTS comprising a T11A mutant (FIG. 5A), a D27G mutant (FIG. 5B), a M96I mutant (FIG. 5C), a G97D mutant (FIG. 5D), a K101R mutant (FIG. 5E), a control using an MG72 O-tRNA (FIG. 5F), and a T11A; D27G; M96I; G97D, K101R mutant (FIG. 5G).

FIG. 6A and FIG. 6B are graphs showing summary scores for candidate O-RS.

FIG. 7 is a schematic showing the positions of various O-RS substitutions identified herein.

FIG. 8 is a bar graph depicting optical density (OD) normalized GFP fluorescence intensity of the O-RS E9VR and O-RS E9VR_5mut mutants with single additional substitutions, expressed by the SoluProm expression system. OD normalized fluorescence in the presence of AcF, AzF and PaF respectively is presented. Wild type GFP is shown as the positive control (“5637+wt GFPmut2” and “5639+wt GFPmut2”) and fluorescence measured in absence of nsAA was used as the negative control (not shown). Relative read-through efficiency (RRE) and maximum misincorporation frequency (MMF) and presented in the overlaid line graph. The dashed line highlights the performance gain relative to E9VR_5mut.

FIG. 9A and FIG. 9B are bar graphs depicting optical density (OD) normalized GFP fluorescence intensity of the O-RS E9VR and O-RS E9VR_5mut with stacking of privileged single substitutions, expressed by the SoluPro™ expression system. OD normalized fluorescence in the presence of AcF, AzF and PaF respectively is presented. Wild type GFP is shown as the positive control (“5637+wt GFPmut2” and “5639+wt GFPmut2”) and fluorescence measured in absence of nsAA was used as a negative control (not shown). Relative read-through efficiency (RRE) and maximum misincorporation frequency (MMF) and presented in the overlaid line graph. The dashed line highlights the performance gain relative to E9VR_5mut.

FIG. 10 is a bar graph depicting optical density (OD) normalized GFP fluorescence intensity of O-RS E9VR and O-RS E9VR_5mut/backbone/tRNA promoter permutation and additional single mutation stacking, expressed by the SoluProm expression system. OD normalized fluorescence in the presence of AcF, AzF and PaF respectively is presented. Four (4) OTSs (F12_E9VR, MG72_E9VR, MG72_E9VR_5mut and MG72_E9VR 6mut), 2 promoters for tRNA (plpp and proK), 3 plasmid backbones (pBK, pEV and pUL) were permutated and GFP with 2 amber codons was used to assess read-through efficiency (RRE) and maximum misincorporation frequency (MMF). The dashed line highlighted the performance gain relative to E9VR_5mut in pBK backbone. Wild type GFP was used as a positive control and fluorescence measured in absence of nsAA was used as a negative control (not shown). RRE MMF are presented in the overlaid line graph.

FIG. 11 is an illustration of a plasmid map for the pBK_MG72_E9VR_6mut plasmid used for the expression of O-RS sequences.

FIG. 12 is an illustration of a plasmid map for the pEV_MG72_E9VR_6mut plasmid used for the expression of O-RS sequences.

FIG. 13 is an illustration of a plasmid map for the pUL_MG72_E9VR_6mut plasmid used for the expression of O-RS sequences.

### DETAILED DESCRIPTION

### Definitions

Terms used in the claims and specification are defined as set forth below unless otherwise specified.

As used herein, the term “orthogonal” refers to a molecule (e.g., an orthogonal tRNA (O-tRNA) and/or an orthogonal aminoacyl tRNA synthetase (O-RS)) that is used with reduced efficiency by a system of interest (e.g., a translational system, e.g., a cell) or that fails to function with endogenous components of the cell. In the context of tRNAs and aminoacyl-tRNA synthetases, orthogonal refers to the inability or reduced efficiency, e.g., less than 20% efficient, less than 10% efficient, less than 5% efficient, or e.g., less than 1% efficient, of an orthogonal tRNA and/or orthogonal RS to function in the translation system of interest. The orthogonal molecule lacks a functional endogenous complementary molecule in the cell. For example, an orthogonal tRNA in a translation system of interest is aminoacylated by any endogenous RS of a translation system of interest with reduced or even zero efficiency, when compared to aminoacylation of an endogenous tRNA by an endogenous RS. In another example, an orthogonal RS aminoacylates any endogenous tRNA in the translation system of interest with reduced or even zero efficiency, as compared to aminoacylation of the endogenous tRNA by an endogenous RS. A second orthogonal molecule can be introduced into the cell that functions with the first orthogonal molecule. For example, an orthogonal tRNA/RS pair includes introduced complementary components that function together in the cell with an efficiency (e.g., about 50% efficiency, about 60% efficiency, about 70% efficiency, about 75% efficiency, about 80% efficiency, about 85% efficiency, about 90% efficiency, about 95% efficiency, or about 99% or more efficiency) to that of a tRNA/RS standard amino acid pair.

The term “cognate” refers to components that function together, e.g., a tRNA and an aminoacyl-tRNA synthetase. The components can also be referred to as being complementary.

The term “aminoacylates” refers to transferring of an amino acid to a tRNA by an amino-acyl tRNA synthetase.

The term “preferentially aminoacylates” refers to an efficiency, e.g., about 70% efficient, about 75% efficient, about 80% efficient, about 85% efficient, about 90% efficient, about 95% efficient, or about 99% or more efficient, at which an O-RS aminoacylates an O-tRNA with a selected amino acid, e.g., an nsAA, compared to the O-RS aminoacylating a naturally occurring tRNA or a starting material used to generate the O-tRNA. The nsAA is then incorporated into a growing polypeptide chain with high fidelity, e.g., at greater than about 70% fidelity, at greater than about 75% fidelity, at greater than about 80% fidelity, at greater than about 85% fidelity, at greater than about 90% fidelity, greater than about 95% fidelity, or greater than about 99% fidelity.

The term “selector codon” refers to codons recognized by the O-tRNA in the translation process and not recognized by an endogenous tRNA. The O-tRNA anti codon loop recognizes the selector codon on the mRNA and incorporates its non-standard amino acid (nsAA), at this site in the polypeptide. Selector codons can include but are not limited to, e.g., nonsense codons, such as, stop codons, including but not limited to, amber, ochre, and opal codons; four or more base codons; rare codons; codons derived from natural or unnatural base pairs and/or the like. For a given system, a selector codon can also include one of the natural three base codons, wherein the endogenous system does not use (or rarely uses) said natural three base codon. For example, this includes a system that is lacking a tRNA that recognizes the natural three base codon, and/or a system wherein the natural three base codon is a rare codon.

The term “non-standard amino acid” (nsAA) refers to any amino acid not naturally occurring in proteins (e.g., a non-naturally occurring modified amino acid or amino acid analogue). In other words, the non-standard amino acid are amino acids other than selenocysteine and/or pyrrolysine and the following twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

Abbreviations used in this application include the following: non-standard amino acid (nsAA), transfer RNA (tRNA), orthogonal tRNA (O-tRNA), and orthogonal amino acyl tRNA synthetase (O-RS).

The term “translation system” refers to the components necessary to incorporate a naturally occurring amino acid into a growing polypeptide chain (protein). Components of a translation system can include, e.g., ribosomes, tRNA's, synthetases, mRNA and the like. The components of the present disclosure can be added to an in vitro or in vivo translation system. Examples of translation systems include but are not limited to, a non-eukaryotic cell, e.g., a bacterium (such as E. coli), a eukaryotic cell, e.g., a yeast cell, a mammalian cell, a plant cell, an algae cell, a fungus cell, an insect cell, a cell-free translational system e.g., a cell lysate, and/or the like.

The term percent “identity,” in the context of two or more polypeptide or nucleic acid sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/). It must be noted that, as used in the specification and the appended claims, the singular forms “a,” “an” and “the” include plural referents unless the context clearly dictates otherwise.

This disclosure provides for tRNAs and the corresponding aminoacyl tRNA synthetases for efficient production of proteins containing non-standard amino acids. These engineered orthogonal tRNA (O-tRNA)/orthogonal aminoacyl tRNA synthetase (O-RS) pair, i.e., Orthogonal Translation Systems (OTSs), can be used to incorporate an nsAA in a specific position in a growing polypeptide in response to a selector codon that is recognized by the tRNA. This disclosure provides for Orthogonal Translation Systems (OTSs) with superior fidelity and efficiency in nsAA incorporation compared to known systems.

Orthogonal Amino-Acyl tRNA Synthetases (O-RS) and Methods of Identifying Same

Described herein are orthogonal aminoacyl-tRNA synthetases (O-RS) that aminoacylate orthogonal tRNAs with nsAAs. In certain embodiments, the O-RS is derived from M. jannaschii tyrosyl-tRNA synthetase. In certain embodiments, the O-RS is derived from (is a variant of) a synthetase having the amino acid sequence set forth in SEQ ID NO: 35 or 39. In certain embodiments, the O-RS comprises an amino acid sequence consisting of the sequence set forth in SEQ ID NO: 46, SEQ ID NO: 48; or SEQ ID NO: 50.

In certain aspects, the disclosure relates to an orthogonal tRNA synthetase (O-RS) comprising a substitution of at least one, at least two, at least three, at least four, at least five, at least six, at least 7, at least 8 or at least 9 of the following residues as compared to a wild-type M. jannaschii tRNA synthetase (SEQ ID NO:45): (a) T11, (b) I15, (c) D27, (d) M96, (e) G97, (f) K101R, (g) G158, (h) R257, (i) F261, (j) E272, (k) P284, (1) M285, and (m) R286, but does not comprise SEQ ID NO: 35. In certain aspects, the disclosure relates to an orthogonal tRNA synthetase (O-RS) comprising a substitution of at least one of the following residues as compared to a wild-type M. jannaschii tRNA synthetase (SEQ ID NO:45): (a) T11, (b) I15, (c) D27, (d) M96, (e) G97, (f) K101R, and (g) G158. In certain embodiments, the O-RS comprises at least 85% sequence identity to SEQ ID NO: 45 but does not comprise SEQ ID NO: 35. For example, in certain embodiments, the O-RS comprises at least 85%, at least 90%, at least 95%, at least 98%, at least 99%, or at least 99.5% sequence identity to SEQ ID NO: 45 but does not comprise SEQ ID NO: 35. In certain embodiments, the O-RS comprises an amino acid sequence comprising 1, up to 2, up to 3, up to 4, up to 5, up to 6, up to 7, up to 8, up to 9, or up to 10 substitutions as compared to SEQ ID NO: 45 but does not comprise SEQ ID NO: 35.

In certain embodiments, the O-RS further comprises a substitution at D286. In certain embodiments, the substitution at D286 is a D286R, D286F, D286W, D286H, D286K or a D286Y substitution. In certain embodiments, the O-RS comprises at least one of the following substitutions: (a) T11A, T11V, T11I, T11L, or T11G; (b) I15V, I15A, I15L, or I15G; (c) D27G, D27A, D27V, D27I, or D27L; (d) M96I, M96A, M96V, M96L, or M96G; (e) G97D or G97E; (f) K101R, K101H, or K101K, and (g) G158 D or G158E. In certain embodiments, the O-RS comprises at least one of the following substitutions: (a) T11A, (b) I15V, (c) D27G, (d) M96I, (e) G97D, (f) K101R and (g) G158D.

In certain embodiments, the O-RS a substitution of at least one of the following residues as compared to a wild-type M. jannaschii tRNA synthetase (SEQ ID NO:45): (a) T11, (b) I15, (c) D27, (d) M96, (e) G97, and (f) K101. In certain embodiments, the orthogonal tRNA synthetase comprises at least 85% sequence identity to SEQ ID NO: 45 but is not identical to SEQ ID NO: 35. In certain embodiments, the O-RS comprises at least one of the following substitutions: (a) T11A, T11V, T11I, T11L, or T11G; (b) I15V, I15A, I15L, or I15G; (c) D27G, D27A, D27V, D27I, or D27L; (d) M96I, M96A, M96V, M96L, or M96G; (e) G97D or G97E; and (f) K101R, or K101H. In certain embodiments, the O-RS comprises at least one of the following substitutions: (a) T11A, (b) I15V, (c) D27G, (d) M96I, (e) G97D, and (f) K101R.

In certain embodiments, the O-RS comprises substitutions at residues I15 and D286, wherein the residues are numbered according to the sequence of a wild-type M. jannaschii tRNA synthetase (SEQ ID NO:45). In certain embodiments, the O-RS comprises at least one of the substitutions I15V and D286R. In certain embodiments, the O-RS comprises the substitutions I15V and D286R. In certain embodiments, the O-RS comprises I15V and D286R and an additional substitution of at least one of the following residues as compared to a wild-type M. jannaschii tRNA synthetase (SEQ ID NO:45): a) R257, b) F261, c) P284, d) M285; e) D286 and f) G158. In certain embodiments, the O-RS comprises the substitutions T11A, I15V, D27G, M96I, G97D, K101R, and D286R. In certain embodiments, the O-RS comprises the substitutions T11A, I15V, D27G, M96I, G97D, K101R, F261P, P284S, and D286R.

In certain embodiments, the O-RS comprises at least one of the following substitutions:
(a) R257W, R257F, R257Y, or R257H,
(b) F261P,
(c) P284S, P284A, P284G, P284C, or P284V,
(d) M285D, or M285E,
(e) D286Y, D286W, D286F, or D286H, and
(f) G158D or G158E.

In certain embodiments, the O-RS comprises at least one of the following substitutions:
(a) R257W,
(b) F261P,
(c) P284S,
(d) M285D,
(e) D286Y, and
(f) G158D.

In certain embodiments, the O-RS further comprises a substitution at amino acid 286. In certain embodiments, the substitution at D286 is a D286F, D286W, D286H, D286K, D286V, or a D286Y substitution. In certain embodiments, the substitution at D286 is D286Y.

In certain embodiments, the O-RS comprises an additional substitution of at least one of the following residues as compared to a wild-type M. jannaschii tRNA synthetase (SEQ ID NO:45): 257, 261 and 284. In certain embodiments, the O-RS comprises at least one of the following substitutions: R257W, R257F, R257Y, R257H, F261P, E272V, P284S, P284A, P284G, P284C, P284V, M285D, M285F, R286V, and R286Y.

In certain embodiments, the O-RS comprises the mutations: I15V, D286R, T11A, D27G, M96I, G97D, K101R, R257W, and P284S.

In certain embodiments, the O-RS comprises the mutations: I15V, D286R, T11A, D27G, M96I, G97D, K101R, R257W and F261P.

In certain embodiments, the O-RS comprises the mutations: I15V, D286R, T11A, D27G, M96I, G97D, K101R, R257W, F261P, and P284S.

In certain embodiments, the O-RS comprises the mutations: I15V, T11A, D27G, M96I, G97D, K101R, R257W, F261P and D286Y.

In certain embodiments, the O-RS comprises the mutations: I15V, T11A, D27G, M96I, G97D, K101R, and D286Y.

In certain embodiments, the O-RS comprises the mutations: I15V, T11A, D27G, M96I, G97D, K101R, F261P, and D286Y.

In certain embodiments, the O-RS comprises the mutations: I15V, D286R, T11A, D27G, M96I, G97D, K101R, and M285D.

In certain embodiments, the O-RS comprises the mutations: I15V, D286R, T11A, D27G, M96I, G97D, K101R, and M285F.

In certain embodiments, the O-RS comprises the mutations: I15V, T11A, D27G, M96I, G97D, K101R, and R286Y.

In certain embodiments, the O-RS comprises the mutations: I15V, T11A, D27G, M96I, G97D, K101R, and R286V.

In certain embodiments, the O-RS comprises the mutations: I15V, T11A, D27G, M96I, G97D, K101R, and R257W.

In certain embodiments, the O-RS comprises the mutations: I15V, T11A, D27G, M96I, G97D, K101R, and P284S.

In certain embodiments, the O-RS comprises the mutations: I15V, T11A, D27G, M96I, G97D, K101R, and F261P.

In certain embodiments, the O-RS comprises the mutations: I15V, T11A, D27G, M96I, G97D, K101R, and E272V.

In certain embodiments, the O-RS further comprises one or more substitutions selected from K90Q, I176L, R257W, P258A, F261P, E272V, H283L, H283T, P284V, P284S, M285F, M285D, D286Y, and D286V.

In certain embodiments, the O-RS comprises an amino acid sequence that is at least 85% identical to a sequence selected from SEQ ID NOs: 39-44 and 46-56. In certain embodiments, the amino acid sequence is at least 90% identical, at least 91% identical, at least 92% identical, at least 93% identical, at least 94% identical, at least 95% identical, at least 96% identical, at least 97% identical, at least 98% identical, or at least 98.7% identical to a sequence selected from SEQ ID NOs: 39-44 and 46-56. In certain embodiments, the O-RS comprises an amino acid sequence comprising 1, up to 2, up to 3, up to 4, up to 5, up to 6, up to 7, up to 8, up to 9, or up to 10 additional substitutions as compared to an amino acid sequence selected from SEQ ID NOs: 39-44 and 46-56. In certain embodiments, the amino acid sequence is selected from SEQ ID NOs: 39-44 and 46-56.

In certain embodiments, the O-RS comprises a conservative substitution relative to an O-RS sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix). In certain embodiments, the O-RS comprises an amino acid sequence comprising 1, up to 2, up to 3, up to 4, up to 5, up to 6, up to 7, up to 8, up to 9, or up to 10 conservative substitutions as compared to SEQ ID NO: 45. In certain embodiments, the O-RS comprises an amino acid sequence comprising 1, up to 2, up to 3, up to 4, up to 5, up to 6, up to 7, up to 8, up to 9, or up to 10 conservative substitutions as compared to any one of SEQ ID NOs: 39-44 and 46-56.

In certain embodiments, the O-RS preferentially aminoacylates the O-tRNAs with nsAAs. The term “preferentially aminoacylates” refers to an efficiency, e.g., about 70% efficient, about 75% efficient, about 80% efficient, about 85% efficient, about 90% efficient, about 95% efficient, or about 99% or more efficient, at which an O-RS aminoacylates an O-tRNA with a selected amino acid, e.g., an nsAA, compared to the O-RS aminoacylating a naturally occurring tRNA. In certain embodiments, efficiency is determined by average read through efficiency “RRE”. In certain embodiments, the relative readthrough efficiency (RRE) of the TAG codon is the GFP/RFP fluorescence ratio for the mcherryTAG assay plasmid divided by the GFP/RFP fluorescence ratio for the mcherryTAC control plasmid.

In certain embodiments, the nsAA is then incorporated into a growing polypeptide chain with high fidelity, e.g., at greater than about 70% fidelity for a given selector codon, at greater than about 75% fidelity for a given selector codon, at greater than about 80% fidelity for a given selector codon, at greater than about 85% fidelity for a given selector codon, at greater than about 90% fidelity for a given selector codon, greater than about 95% fidelity for a given selector codon, or greater than about 99% fidelity for a given selector codon. In certain embodiments, fidelity is determined by average maximum misincorporation frequency “MMF”. In certain embodiments, the maximum misincorporation frequency (MMF), is calculated by dividing the RRE when nsAA was not added to the growth media by the RRE when nsAA is present.

The O-RSs described herein can be derived from a variety of organisms, e.g., non-vertebrate organisms, such as a prokaryotic organism (e.g., E. coli, Bacillus stearothermophilus, or the like), or an archaebacterium, or e.g., a vertebrate organism. In certain embodiments, the O-RS is derived from archaea M. jannaschii.

In certain embodiments, the O-RS has one or more improved or enhanced enzymatic properties for the nsAAs as compared to a natural amino acid. For example, the improved or enhanced properties for the nsAA as compared to a natural amino acid include any of e.g., a higher Km, a lower Km, a higher kcat, a lower kcat, a lower kcat/km, a higher kcat/km, etc.

The disclosure further relates to methods for identifying an orthogonal aminoacyl-tRNA synthetase (O-RS), e.g., an O-RS, for use with an O-tRNA, are also a feature of the present invention. For example, a method includes subjecting to positive selection a population of cells of a first species, where the cells each comprise: 1) a member of a plurality of aminoacyl tRNA synthetases (RSs), where the plurality of RSs comprise mutant RSs, RSs derived from a species other than the first species, or both mutant RSs and RSs derived from a species other than the first species; 2) the orthogonal tRNA (O-tRNA) from a second species; and 3) a polynucleotide that encodes a positive selection marker and comprises at least one selector codon. Cells are selected or screened for those that show an enhancement in suppression efficiency compared to cells lacking or with a reduced amount of the member of the plurality of RSs. In certain embodiments, “suppression efficiency” refers to the ratio of accumulated full-length soluble protein containing a nsAA to its natural counterpart. Because the OTS suppresses the translational terminating activity of the release factor RF1 and reads through the amber codon, “suppression efficiency,” in certain embodiments, can be defined as the ratio of accumulated full-length soluble protein containing the nsAA to gross protein related to the target, including full-length modified protein and the prematurely truncated protein.

Cells having an enhancement in suppression efficiency comprise an active RS that aminoacylates the O-tRNA. A level of aminoacylation (in vitro or in vivo) by the active RS of a first set of tRNAs from the first species is compared to the level of aminoacylation (in vitro or in vivo) by the active RS of a second set of tRNAs from the second species. The level of aminoacylation can be determined by a detectable substance (e.g., a labeled amino acid or unnatural amino acid). The active RS that more efficiently aminoacylates the second set of tRNAs compared to the first set of tRNAs is selected, thereby providing the orthogonal aminoacyl-tRNA synthetase for use with the O-tRNA. An O-RS, e.g., an O-RS, identified by the method is also a feature of the present invention.

Orthogonal tRNA (O-tRNA)

Described herein are orthogonal transfer RNAs (O-tRNAs) that are aminoacylated with a non-standard amino acid (nsAA). The O-tRNA mediates incorporation of an nsAA into a protein that is encoded by a polynucleotide that comprises a selector codon that is recognized by the O-tRNA.

In certain aspects, described herein are orthogonal tRNAs (O-tRNA) comprising a nucleic acid sequence at least 85% identical to the sequence set forth in SEQ ID NO: 1 and comprising a deletion of the cytosine located at nucleic acid position 16 of the O-tRNA, wherein the nucleic acid positions correspond to the sequence set forth in SEQ ID NO: 1; and wherein the O-tRNA is capable of being aminoacylated with at least one non-standard amino acid (nsAA) by an orthogonal aminoacyl tRNA synthetase (O-RS). In certain embodiments, the nucleic acid sequence is at least 90% identical, at least 91% identical, at least 92% identical, at least 93% identical, at least 94% identical, at least 95% identical, at least 96% identical, at least 97% identical, at least 98% identical, or at least 98.7% identical to the sequence set forth in SEQ ID NO: 1. In certain embodiments, the O-tRNA comprises an adenine at nucleic acid position 53 and a uracil at nucleic acid position 63, wherein the nucleic acid positions correspond to the sequence set forth in SEQ ID NO: 1. In certain embodiments, the O-tRNA comprises a nucleic acid sequence set forth in SEQ ID NO: 2. In certain embodiments, the O-tRNA comprises a cytosine at amino acid positions 3 and 6; a uracil at nucleic acid position 7, an adenosine at nucleic acid position 67, and a guanine at nucleic acid positions 68 and 71, wherein the nucleic acid positions correspond to the sequence set forth in SEQ ID NO: 1. In certain embodiments, the O-tRNA comprises a nucleic acid sequence consisting of the sequence set forth in SEQ ID NO: 3. In certain embodiments, the O-tRNA comprises a nucleic acid sequence consisting of the sequence set forth in SEQ ID NO: 4. In certain embodiments, the O-tRNA comprises the sequence CAG-AGGGCAG (SEQ ID NO: 74) at nucleic acid positions 13 to 23, wherein the nucleic acid positions correspond to the sequence set forth in SEQ ID NO: 1.

In certain aspects, the disclosure relates to an O-tRNA comprising a nucleic acid sequence consisting of a sequence set forth in SEQ ID NO: 36, wherein the sequence does not comprise SEQ ID NO: 1, SEQ ID NO: 37, or SEQ ID NO: 38.

SEQ ID NO: 36 provides a consensus sequence as follows: CCX₁X₂X₃X₄X₅UAGUUCAGX₆AGGGCAGAACGGCGGACUCUAAAUCCGCAX₇GX₈C X₉X₁₀X₁₁X₁₂GUCAAAUCX₁₃X₁₄X₁₅X₁₆X₁₇X₁₈X₁₉X₂₀X₂₁GGACCA; wherein X₁ is C or G; X₂ is A or G; X₃ is A, U or C; X₄ is C or G; X₅ is U or G; X₆ is C or del; X₇ is G or U; X₈ is G or U; X₉ is A or G; X₁₀ is G or C; X₁ is G, C, A or U; X₁₂ is G or A; X₁₃ is C or U; X₁₄ is G, C, A or U; X₁₅ is G or C; X₁₆ is U or C; X₁₇ is A or C; X₁₈ is G or C; X₁₉ is A, G or U; X₂₀ is U or C; and X₂₁ is C or G.

In certain embodiments, the O-tRNA comprises a cytosine at position 3. In certain embodiments, the O-tRNA comprises an adenine at position 4. In certain embodiments, the O-tRNA comprises a uracil at position 5. In certain embodiments, the O-tRNA comprises a cytosine at position 6. In certain embodiments, the O-tRNA comprises a uracil at position 7. In certain embodiments, the O-tRNA comprises a guanine at position 46. In certain embodiments, the O-tRNA comprises a uracil at position 48. In certain embodiments, the O-tRNA comprises an adenine at position 50. In certain embodiments, the O-tRNA comprises a guanine at position 51. In certain embodiments, the O-tRNA comprises an adenine at position 53. In certain embodiments, the O-tRNA comprises a uracil at position 63. In certain embodiments, the O-tRNA comprises a cytosine at position 64. In certain embodiments, the O-tRNA comprises a cytosine at position 65. In certain embodiments, the O-tRNA comprises a uracil at position 66. In certain embodiments, the O-tRNA comprises an adenine at position 67. In certain embodiments, the O-tRNA comprises a guanine at position 68. In certain embodiments, the O-tRNA comprises an adenine or a uracil at position 69. In certain embodiments, the O-tRNA comprises a uracil at position 70. In certain embodiments, the O-tRNA comprises a guanine at position 71. In certain embodiments, the O-tRNA comprises a cytosine at position 3, a cytosine at position 6, and a uracil at position 7. In certain embodiments, the O-tRNA comprises a guanine at position 46 and a uracil at position 48. In certain embodiments, the O-tRNA comprises an adenine at position 67, a guanine at position 68, and a guanine at position 71.

In certain embodiments, the O-tRNA comprises an adenine at nucleic acid position 52 and a uracil at nucleic acid position 62; wherein the nucleic acid positions correspond to the sequence set forth in SEQ ID NO: 1. In certain embodiments, the O-tRNA comprises an guanine at nucleic acid position 52 and a cytosine at nucleic acid position 62; wherein the nucleic acid positions correspond to the sequence set forth in SEQ ID NO: 1. In certain embodiments, the O-tRNA comprises an guanine at nucleic acid position 51 and a cytosine at nucleic acid position 65; wherein the nucleic acid positions correspond to the sequence set forth in SEQ ID NO: 1. In certain embodiments, the O-tRNA comprises an cytosine at nucleic acid positions 3 and 6, a uracil at nucleic acid position 7, an adenine at nucleic acid position 66, and guanines at nucleic acid positions 67 and 70; wherein the nucleic acid positions correspond to the sequence set forth in SEQ ID NO: 1. In certain embodiments, the O-tRNA comprises a uracil at nucleic acid positions 5, and 7, an adenine at nucleic acid positions 67 and 69; wherein the nucleic acid positions correspond to the sequence set forth in SEQ ID NO: 1. In certain embodiments, the O-tRNA comprises an adenine at nucleic acid positions 4 and 67, a uracil at nucleic acid positions 7 and 70, an cytosine at nucleic acid position 6 and a guanine at nucleic acid position 68; wherein the nucleic acid positions correspond to the sequence set forth in SEQ ID NO: 1. In certain embodiments, the O-tRNA comprises an adenine at nucleic acid position 5, and a uracil at nucleic acid positions 69 and 70; wherein the nucleic acid positions correspond to the sequence set forth in SEQ ID NO: 1. In certain embodiments, the O-tRNA comprises a uracil at nucleic acid position 48; wherein the nucleic acid positions correspond to the sequence set forth in SEQ ID NO: 1. In certain embodiments, the O-tRNA comprises a guanine at nucleic acid position 51; wherein the nucleic acid positions correspond to the sequence set forth in SEQ ID NO: 1. In certain embodiments, the O-tRNA comprises a guanine at nucleic acid position 46 and a uracil at nucleic acid position 48; wherein the nucleic acid positions correspond to the sequence set forth in SEQ ID NO: 1. In certain embodiments, the O-tRNA comprises a guanine at nucleic acid position 51 and a uracil at nucleic acid position 48; wherein the nucleic acid positions correspond to the sequence set forth in SEQ ID NO: 1. In certain embodiments, the O-tRNA comprises a guanine at nucleic acid positions 46 and 51 and a uracil at nucleic acid position 48; wherein the nucleic acid positions correspond to the sequence set forth in SEQ ID NO: 1.

In certain embodiments, the O-tRNAs disclosed herein comprise one or more mutations compared to the wild-type M. jannaschii tyrosyl-tRNA (SEQ ID NO: 32); or one or more mutations compared to the F12 O-tRNA sequence (SEQ ID NO: 1), the F13 O-tRNA sequence (SEQ ID NO: 37), or the F14 O-tRNA sequence (SEQ ID NO: 38). For example, in certain embodiments, nucleotides residing in the stem of the T loop of the O-tRNA are mutated, e.g. G53 and C63 are mutated to A52 and U62; wherein the nucleic acid positions correspond to the sequence set forth in SEQ ID NO: 1. In certain embodiments, paired nucleotides residing in the stem of the T loop of the O-tRNA, i.e., C52 and G64, or U52 and A64, or A52 and U64 are mutated to G52 and C62; wherein the nucleic acid positions correspond to the sequence set forth in SEQ ID NO: 1. In certain embodiments, nucleotides residing in the stem of the T loop of the O-tRNA, i.e., C51 and G65, are mutated to G51 and C65; wherein the nucleic acid positions correspond to the sequence set forth in SEQ ID NO: 1. In certain embodiments, nucleotides residing in the stem of the acceptor stem of the O-tRNA, i.e., G3, G6, G7, C67, C68 and C71 are mutated to C3, C6, U7, A66, G67 and G70; wherein the nucleic acid positions correspond to the sequence set forth in SEQ ID NO: 1. In certain embodiments, nucleotides residing in the stem of the acceptor stem of the O-tRNA, i.e., C5, G7, C67 and G69 are mutated to U5, U7, A67 and A69; wherein the nucleic acid positions correspond to the sequence set forth in SEQ ID NO: 1. In certain embodiments, nucleotides residing in the stem of the acceptor stem of the O-tRNA, i.e., G4, G6, G7, C67, C68 and C70 are mutated to A4, C6, U7, A67, G68 and U70; wherein the nucleic acid positions correspond to the sequence set forth in SEQ ID NO: 1. In certain embodiments, nucleotides residing in the stem of the acceptor stem of the O-tRNA, i.e., C5, G69 and C70 are mutated to A5, U69 and U70; wherein the nucleic acid positions correspond to the sequence set forth in SEQ ID NO: 1. In certain embodiments, a nucleotide residing in the variable loop of the O-tRNA, i.e., G48 is mutated to U48; wherein the nucleic acid positions correspond to the sequence set forth in SEQ ID NO: 1. In certain embodiments, a nucleotide residing in the variable loop, i.e., C51 is mutated to G51; wherein the nucleic acid positions correspond to the sequence set forth in SEQ ID NO: 1. In certain embodiments, a nucleotide residing in the variable loop, i.e., U46, and G48 are mutated to G46 and U48; wherein the nucleic acid positions correspond to the sequence set forth in SEQ ID NO: 1. In certain embodiments, a nucleotides residing in the variable loop, i.e., G48 and C51 are mutated to U48 and G51; wherein the nucleic acid positions correspond to the sequence set forth in SEQ ID NO: 1. In certain embodiments, a nucleotide residing in the variable loop, i.e., U46, G48, and C51 are mutated to G46, U48, and G51.

In certain embodiments, the O-tRNA comprises a nucleic acid sequence consisting of the sequence set forth in SEQ ID NO. 2-16.

A tRNA may be aminoacylated with a desired amino acid by any method or technique, including but not limited to, chemical or enzymatic aminoacylation. Aminoacylation may be accomplished by aminoacyl tRNA synthetases or by other enzymatic molecules, including but not limited to, ribozymes. The term “ribozyme” is interchangeable with “catalytic RNA.” Thus, in certain embodiments, the O-tRNA is chemically aminoacylated. In certain embodiments, the O-tRNA is enzymatically aminoacylated. In certain embodiments, the O-tRNA is enzymatically aminoacylated by a ribozyme.

The O-tRNAs described herein can be derived from a variety of organisms, e.g., non-vertebrate organisms, such as a prokaryotic organism (e.g., E. coli, Bacillus stearothermophilus, or the like), or an archaebacterium, or e.g., a vertebrate organism. In certain embodiments, the O-tRNA is derived from an archaeal tRNA. In certain embodiments, the O-tRNA is derived from a Methanococcus jannaschii tRNA.

In an aspect, the O-tRNA has an anticodon that will pair with a selector codon. In certain embodiments, the selector codon is the amber stop codon (TAG), thus allowing the incorporation of the nsAA at the TAG codon. Because the TAG codon naturally functions as a stop codon through recognition by Release Factor I (which terminates protein synthesis), competition ensues between incorporation of the nsAA and termination of protein synthesis.

The functionality of OTSs can be further improved by knocking out or reducing the function of the Release Factor I that competes with nsAA incorporation at the amber codon, engineering a protein elongation factor to better accommodate the OTS tRNA, and new methods for OTS directed evolution. Accordingly, in certain embodiments the OTS has reduced expression of Release Factor 1 (e.g., about 15-50% less, about 25-75% less, about 50-100% less or about 75-100% less) compared to an otherwise identical wild-type cell. In certain embodiments, the OTS has no Release Factor I.

In certain embodiments, the O-tRNA is post-transcriptionally modified when expressed in a cell.

Orthogonal Translation Systems (OTSs)

This disclosure describes orthogonal translation systems (OTSs) that comprise an orthogonal aminoacyl-tRNA synthetase (O-RS) and an orthogonal tRNA (O-tRNA) described herein. For example, an OTS can comprise an O-RS comprising a nucleic acid having a sequence selected from SEQ ID NO: 46, SEQ ID NO: 48; and SEQ ID NO: 50 and an O-tRNA comprising a nucleic acid having the sequence SEQ ID NO: 2. In certain embodiments, the OTS further comprises an nsAA described herein. Optionally, an nsAA is provided exogenously to the OTS. Alternately, e.g., where the OTS is a cell, the non-standard amino acid can be biosynthesized by the OTS. In certain embodiments, the OTS further comprises a mutant EF-Tu. In certain embodiments, Release Factor I has been removed or modified, or Release Factor I expression has been reduced in the OTS. In certain embodiments, the OTS comprises an engineered or modified protein elongation factor to better accommodate the OTS tRNA during translation (e.g., increase efficacy and/or fidelity). In certain embodiments, the modified elongation factor is an EF-Tu as described in Haruna K. et al. Nucleic Acids Research, Vol 42, Issue 15, 2 Sep. 2014, 9976-9983.

The individual components of an O-tRNA/O-RS pair can be derived from the same organism or different organisms. In an embodiment, the O-tRNA/O-RS pair is from the same organism. Alternatively, the O-tRNA and the O-RS of the O-tRNA/O-RS pair are from different organisms. In certain embodiments, the O-tRNA and the O-RS are derived from archaea. In certain embodiments, the O-tRNA and the O-RS are derived from M. jannaschii.

In certain embodiments, the disclosure provides a cell comprising an orthogonal aminoacyl-tRNA synthetase (O-RS), an orthogonal tRNA (O-tRNA), a nucleic acid that comprises a polynucleotide that encodes a polypeptide of interest, and optionally a nsAA described herein. In an aspect, the polynucleotide comprises at least one selector codon that is recognized by the O-tRNA. In an aspect, the O-RS preferentially aminoacylates the orthogonal tRNA (O-tRNA) with the nsAA in the cell, and the cell produces the polypeptide of interest in the absence of the nsAA, with a yield that is, e.g., less than 30%, less than 20%, less than 15%, less than 10%, less than 5%, less than 2.5%, less than 1%, etc., of the yield of the polypeptide in the presence of the nsAA.

Translation systems may be cellular or cell-free, and may be prokaryotic or eukaryotic. Cellular translation systems include, but are not limited to, whole cell preparations such as permeabilized cells or cell cultures wherein a desired nucleic acid sequence can be transcribed to mRNA and the mRNA translated. Cell-free translation systems are commercially available and many different types and systems are well known.

In certain embodiments, this disclosure provides a cell-free OTS comprising an orthogonal aminoacyl-tRNA synthetase (O-RS), an orthogonal tRNA (O-tRNA), a nucleic acid that comprises a polynucleotide that encodes a polypeptide of interest, and an nsAA.

Examples of cell-free systems include, but are not limited to, prokaryotic lysates such as Escherichia coli lysates, and eukaryotic lysates such as wheat germ extracts, insect cell lysates, rabbit reticulocyte lysates, rabbit oocyte lysates and human cell lysates. Eukaryotic extracts or lysates may be preferred when the resulting protein is glycosylated, phosphorylated or otherwise modified because many such modifications are only possible in eukaryotic systems. Some of these extracts and lysates are available commercially. Membranous extracts, such as the canine pancreatic extracts containing microsomal membranes, are also available which are useful for translating secretory proteins.

Reconstituted translation systems may also be used. Mixtures of purified translation factors have also been used successfully to translate mRNA into protein as well as combinations of lysates or lysates supplemented with purified translation factors such as initiation factor-1 (IF-1), IF-2, IF-3 (C or B), elongation factor T (EF-Tu), or termination factors.

Cell-free systems may also be coupled transcription/translation systems wherein DNA is introduced to the system, transcribed into mRNA and the mRNA translated as described in Current Protocols in Molecular Biology (F. M. Ausubel et al. editors, Wiley Interscience, 1993). RNA transcribed in a eukaryotic transcription system may be in the form of heteronuclear RNA (hnRNA) or 5′-end caps (7-methyl guanosine) and 3′-end poly A tailed mature mRNA, which can be an advantage in certain translation systems. For example, capped mRNAs are translated with high efficiency in the reticulocyte lysate system.

Further, a coupled transcription/translation system may be used. Coupled transcription/translation systems allow for transcription of an input DNA into a corresponding mRNA, which is in turn translated by the reaction components. For example, a system that includes a mixture containing E. coli lysate for providing translational components such as ribosomes and translation factors could be used.

Non-Standard Amino Acids (nsAAs)

Non-standard amino acids (nsAAs) are incorporated into polypeptides by the orthogonal translation systems described herein. The generic structure of an alpha amino acid is illustrated by Formula I:

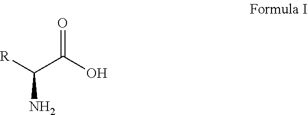

A non-standard amino acid is a non-naturally occurring amino acid, and comprises any structure having Formula I wherein the R group is any substituent other than one used in the twenty natural amino acids, which distinguish them from natural amino acids. For example, R in Formula I may comprise an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, and the like, or any combination thereof.

Other non-standard amino acids include, but are not limited to, amino acids comprising a photoactivatable cross-linker, a spin-labeled amino acid, a fluorescent amino acid, a metal binding amino acid, a metal containing amino acid, a radioactive amino acid, amino acids with novel functional groups, amino acids that covalently or noncovalently interact with other molecules, photocaged amino acids, photoisomerizable amino acids, amino acids comprising biotin or a biotin analogue, glycosylated amino acids such as a sugar substituted serine, other carbohydrate modified amino acids, keto-containing amino acids, amino acids comprising polyethylene glycol or polyether, a heavy atom substituted amino acids, chemically cleavable amino acids, photocleavable amino acids, amino acids with an elongated side chains as compared to natural amino acids, including but not limited to, polyethers or long chain hydrocarbons, including but not limited to, greater than about five or greater than about ten carbons, carbon-linked sugar-containing amino acids, redox active amino acids, amino thio acid containing amino acids, and amino acids comprising one or more toxic moiety.

In certain embodiments, the non-standard amino acid is a derivative of a natural amino acid, such as tyrosine, glutamine, phenylalanine, and the like. In certain embodiments, the non-standard amino acid is a tyrosine analog. In certain embodiments, the tyrosine analogs include para-substituted tyrosines, ortho-substituted tyrosines, and meta-substituted tyrosines, wherein the substituted tyrosine comprises a keto group (including but not limited to, an acetyl group), a benzoyl group, an amino group, a hydrazine, a hydroxyamine, a thiol group, a carboxy group, an isopropyl group, a methyl group, a branched hydrocarbon, a saturated or unsaturated hydrocarbon, an O-methyl group, a polyether group, a nitro group, or the like. Multiply substituted aryl rings are also contemplated. Glutamine analogs include, but are not limited to, α-hydroxy derivatives, γ-substituted derivatives, cyclic derivatives, and amide substituted glutamine derivatives. Example phenylalanine analogs include, but are not limited to, para-substituted phenylalanines, ortho-substituted phenylalanines, and meta-substituted phenylalanines, wherein the Substituent comprises a hydroxy group, a methoxy group, a methyl group, an allyl group, an aldehyde, an azido, an iodo, a bromo, a keto group (including but not limited to, an acetyl group), or the like.

Specific examples of non-standard amino acids include, but are not limited to, a p-acetyl-L-phenylalanine (Formula II), a p-azido-L-phenylalanine (Formula III), a p-propargyl phenylalanine (Formula IV), an O-methyl-L-tyrosine, an L-3-(2-naphthyl)alanine, a 3-methylphenylalanine, an O-4-allyl-L-tyrosine, a 4-propyl-L-tyrosine, a tri-O-acetyl-GlcNAcB-serine, an L-Dopa, a fluorinated phenylalanine, an isopropyl-L-phenylalanine, a p-acyl-L-phenylalanine, a p-benzoyl-L-phenylalanine, an L-phosphoserine, a phosphono serine, a phosphonotyrosine, a p-iodo-phenylalanine, a p-bromophenylalanine, a p-amino-L-phenylalanine, and an isopropyl-L-phenylalanine.

In certain embodiments, the non-standard amino acid is an O-methyl-L-tyrosine. In certain embodiments, the non-standard amino acid is an L-3-(2-naphthyl)alanine. In certain embodiments, the non-standard amino acid is an amino-, an isopropyl-, or an O-allyl-containing phenylalanine analogue. In certain embodiments, the non-standard amino acid is acetyl-phenylalanine (AcF). In certain embodiments, the non-standard amino acid is 4-azido-phenylalanine (AzF). In certain embodiments, the non-standard amino acid is 4-propargyloxyphenylalanine (PaF). In certain embodiments, the non-standard amino acid is 4-aminophenylalanine (AmF). In certain embodiments, the non-standard amino acid is 4-azidomethyl-phenylalanine (mAzF).

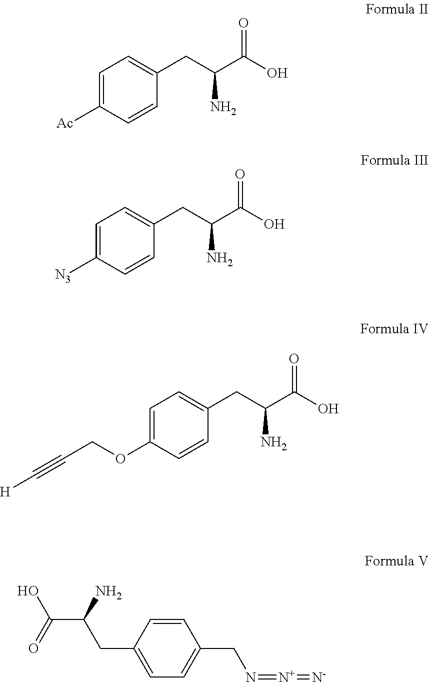 [SMILES: CC(=O)c1ccc(C[C@H](N)C(=O)O)cc1 ; [N-]=[N+]=Nc1ccc(C[C@H](N)C(=O)O)cc1 ; C#CCOc1ccc(C[C@H](N)C(=O)O)cc1 ; [N-]=[N+]=NCc1ccc(C[C@H](N)C(=O)O)cc1]

Nucleic Acids

The present disclosure includes a polynucleotide or set of polynucleotides comprising a nucleic acid sequence of an O-tRNA and/or encoding an O-RS described herein. The present disclosure also includes nucleic acid sequences complementary to an O-tRNA and/or an O-RS described herein.

In addition, this disclosure includes polynucleotides encoding a protein of interest described herein comprising one or more selector codon(s). In certain embodiments, the nucleic acid comprises at least one selector codon, at least two selector codons, at least three selector codons, at least four selector codons, at least five selector codons, at least six selector codons, at least seven selector codons, at least eight selector codons, at least nine selector codons, or even ten or more selector codons.

In an aspect, described herein is a polynucleotide comprising a nucleic acid sequence at least 85% identical to the sequence set forth in SEQ ID NO: 1 and comprising a deletion of the cytosine located at nucleic acid position 16 of the O-tRNA, wherein the nucleic acid positions correspond to the sequence set forth in SEQ ID NO: 1. In certain embodiments, the nucleic acid sequence is at least 90% identical, at least 91% identical, at least 92% identical, at least 93% identical, at least 94% identical, at least 95% identical, at least 96% identical, at least 97% identical, at least 98% identical, or at least 99% identical to the sequence set forth in SEQ ID NO: 1. In an embodiment, the nucleic acid sequence comprises an adenine at nucleic acid position 52 and a thymine or uracil at nucleic acid position 62, wherein the nucleic acid positions correspond to the sequence set forth in SEQ ID NO: 1. In an embodiment, the polynucleotide comprises the nucleic acid sequence consisting of the sequence set forth in SEQ ID NO: 2. In an embodiment, the nucleic acid sequences comprises a cytosine at amino acid positions 3 and 6; a thymine or uracil at nucleic acid position 7, an adenosine at nucleic acid position 66, and a guanine at nucleic acid positions 67 and 70, wherein the nucleic acid positions correspond to the sequence set forth in SEQ ID NO: 1. In an embodiment, the polynucleotide comprises a nucleic acid sequence consisting of the sequence set forth in SEQ ID NO: 3. In an embodiment, the polynucleotide comprises a nucleic acid sequence consisting of the sequence set forth in SEQ ID NO: 4. In an embodiment, the polynucleotide comprises the nucleic acid sequence CAG-AGGGCAG (SEQ ID NO: 74) at nucleic acid positions 13 to 22, wherein the nucleic acid positions correspond to the sequence set forth in SEQ ID NO: 1. In an embodiment, the polynucleotide comprises a nucleic acid sequence consisting of a sequence set forth in any one of SEQ ID NOs: 2-16.

In an aspect, described herein is a polynucleotide comprising a nucleic acid sequence of an O-tRNA comprising a nucleic acid sequence set forth in any one of SEQ ID NOs: 2-31. In an embodiment, the polynucleotides further comprise a nucleic acid sequence complementary to an O-tRNA comprising a nucleic acid sequence set forth in any one of SEQ ID NOs: 2-31.

In an aspect, described herein are polynucleotides comprising a nucleic acid sequence encoding an O-RS comprising an amino acid sequence set forth in any one of SEQ ID NOs: 39-44 and 46-56. In certain embodiments, the polynucleotides further comprise a nucleic acid sequence complementary to a nucleic acid sequence encoding an O-RS comprising an amino acid sequence set forth in any one of SEQ ID NOs: 39-44 and 46-56.

In an aspect, the disclosure relates to a polynucleotide encoding an O-RS comprising a nucleic acid sequence consisting of a sequence set forth in any one of SEQ ID NOs: 57-73. In certain embodiments, the polynucleotide comprises a nucleic acid sequence complementary to the O-RS sequence consisting of a sequence set forth in any one of SEQ ID NOs: 57-73.

In an aspect, described herein is a polynucleotide or set of polynucleotides comprising a nucleic acid sequence of an O-tRNA comprising a nucleic acid sequence set forth in any one of SEQ ID NOs: 2-31 and a nucleic acid sequence encoding the O-RS comprising an amino acid sequence set forth in any one of SEQ ID NOs: 39-44 and 46-56 or a nucleic acid sequence consisting of a sequence set forth in any one of SEQ ID NOs: 57-73.

Vectors

In certain embodiments, a vector (e.g., a plasmid, a cosmid, a phage, a virus, etc.) comprises a polynucleotide described herein. In certain embodiments, the vector is an expression vector. In certain embodiments, the expression vector includes a promoter operably linked to one or more of the polynucleotides described herein. In certain embodiments, a cell comprises a vector that includes a polynucleotide disclosed herein. In certain embodiments, the vector is a plasmid, (e.g., pBK, pEV and pUL). In certain embodiments, the promoter is a plpp or a proK promoter for expression of tRNA.

Cells

In certain embodiments, this disclosure includes cells comprising an O-tRNA, an O-RS, an nsAA, and/or an OTS described herein. The cells described herein include any of, e.g., prokaryotic cells (e.g., Escherichia coli), non-prokaryotic cells, mammalian cells, yeast cells, fungus cells, plant cells, insect cells, etc. In certain embodiments, the cell encodes a mutation in an EF-Tu. In certain embodiments, the cell has reduced expression of Release Factor 1 compared to an otherwise identical wild-type cell.

In certain embodiments, the cells containing the O-tRNA, O-RS, nsAA, and/or OTS described herein are cells as described in U.S. Pat. Nos. 9,617,335, 10,465,197, 10,604,761, and U.S. application Ser. No. 15/261,984, U.S. application Ser. No. 16/871,736, which are incorporated herein by reference in their entireties.

In certain aspects, described herein are cells comprising a nucleic acid that comprises a polynucleotide that encodes a polypeptide of interest, where the polynucleotide comprises a selector codon that is recognized by the O-tRNA. In one aspect, the yield of the polypeptide of interest comprising the nsAA is, e.g., at least 2.5%, at least 5%, at least 10%, at least 25%, at least 30%, at least 40%, 50% or more, of that obtained for the naturally occurring polypeptide of interest from a cell in which the polynucleotide lacks the selector codon. In another aspect, the cell produces the polypeptide of interest in the absence of the nsAA, with a yield that is, e.g., less than 50%, less than 35%, less than 30%, less than 20%, less than 15%, less than 10%, less than 5%, less than 2.5%, etc., of the yield of the polypeptide in the presence of the nsAA.

Compositions that include a cell comprising an orthogonal tRNA (O-tRNA) are also a feature of the invention. Typically, the O-tRNA mediates incorporation of an nsAA into a protein that is encoded by a polynucleotide that comprises a selection codon that is recognized by the O-tRNA in vivo. In one embodiment, the O-tRNA mediates the incorporation of the nsAA into the protein with, e.g., at least 45%, at least 50%, at least 60%, at least 75%, at least 80%, at least 90%, at least 95%, or even 99% or more the efficiency of a tRNA that comprises or is processed in a cell from a polynucleotide sequence as set forth in SEQ ID NOs: 2-16. In another embodiment, the O-tRNA comprises or is processed from a polynucleotide sequence as set forth in SEQ ID NOs: 2-16, or a conservative variation thereof. In yet another embodiment, the O-tRNA comprises a recyclable O-tRNA.

In certain embodiments, the cells described herein have the ability to synthesize proteins that comprise nsAAs in large useful quantities. For example, proteins comprising an nsAA can be produced at a concentration of, e.g., at least 10 μg/liter, at least 50 μg/liter, at least 75 μg/liter, at least 100 μg/liter, at least 200 μg/liter, at least 250 μg/liter, or at least 500 μg/liter or more of protein in a cell extract, a buffer, a pharmaceutically acceptable excipient, and/or the like. In certain embodiments, a composition of the invention includes, e.g., at least 10 μg, at least 50 μg, at least 75 μg, at least 100 μg, at least 200 μg, at least 250 μg, or at least 500 μg or more of protein that comprises an nsAA.

Once a recombinant host cell strain has been established (i.e., one or more expression vectors comprising polynucleotide sequences for the O-tRNA and/or O-RT has been introduced into the host cell and host cells with the proper expression construct are isolated), the recombinant host cell strain is cultured under conditions appropriate for production of the polypeptide of interest. As will be apparent to one of skill in the art, the method of culture of the recombinant host cell strain will be dependent on the nature of the expression construct utilized and the identity of the host cell. Recombinant host cells may be cultured in batch or continuous formats, with either cell harvesting (in the case where the polypeptide of interest accumulates intracellularly) or harvesting of culture supernatant in either batch or continuous formats. For production in prokaryotic host cells, batch culture and cell harvest can be performed. In certain embodiments, fed batch fermentation culturing conditions are used.

Polypeptides Comprising at Least One nsAA

Proteins (or polypeptides of interest) with at least one nsAA are also disclosed herein. In certain embodiments, a protein with at least one nsAA includes at least one post-translational modification. In an embodiment, the at least one post-translational modification comprises attachment of a molecule (e.g., a dye, a polymer, e.g., a derivative of polyethylene glycol, a photocrosslinker, a cytotoxic compound, an affinity label, a derivative of biotin, a resin, a second protein or polypeptide, a metal chelator, a cofactor, a fatty acid, a carbohydrate, a polynucleotide (e.g., DNA, RNA, etc.), etc.) comprising a second reactive group by a [3+2] cycloaddition to the at least one nsAA comprising a first reactive group. For example, the first reactive group is an alkynyl moiety (e.g., in the nsAA p-propargyloxyphenylalanine) (this group is also sometimes refer to as an acetylene moiety) and the second reactive group is an azido moiety. In another example, the first reactive group is the azido moiety (e.g., in the nsAA p-azido-L-phenylalanine) and the second reactive group is the alkynyl moiety. In certain embodiments, a protein of the invention includes at least one nsAA (e.g., a keto nsAA) comprising at least one post-translational modification, where the at least one post-translational modification comprises a saccharide moiety. In certain embodiments, the post-translational modification is made in vivo in a cell. Thus, in certain embodiments, the protein(s) comprising non-standard amino acids that are produced are processed and modified in a cell-dependent manner. This provides for the production of proteins that are stably folded, glycosylated, or otherwise modified by the cell.

In certain embodiments, the protein includes at least one post-translational modification that is made in vivo by a cell, where the post-translational modification is not made by a prokaryotic cell. Examples of post-translational modifications include, but are not limited to, acetylation, acylation, lipid-modification, palmitoylation, palmitate addition, phosphorylation, glycolipid-linkage modification, and the like. In an embodiment, the post-translational modification comprises attachment of an oligosaccharide to an asparagine by a GlcNAc-asparagine linkage (e.g., where the oligosaccharide comprises (GlcNAc-Man)₂-Man-GlcNAc-GlcNAc, and the like). In another embodiment, the post-translational modification comprises attachment of an oligosaccharide (e.g., Gal-GalNAc, Gal-GlcNAc, etc.) to a serine or threonine by a GalNAc-serine, a GalNAc-threonine, a GlcNAc-serine, or a GlcNAc-threonine linkage. In certain embodiments, a protein or polypeptide of the invention can comprise a secretion or localization sequence, an epitope tag, a FLAG tag, a polyhistidine tag, a GST fusion, and/or the like.

Typically, the proteins are, e.g., at least 60%, at least 70%, at least 75%, at least 80%, at least 90%, at least 95%, or even at least 99% or more identical to any available protein (e.g., a therapeutic protein, a diagnostic protein, an industrial enzyme, or portion thereof, and/or the like), and they comprise one or more nsAA. In an embodiment, disclosed herein are composition comprising a protein or polypeptide of interest comprising at least one nsAA and an excipient (e.g., a buffer, a pharmaceutically acceptable excipient, etc.).

Examples of a protein (or polypeptides) of interest include, but are not limited to, e.g., an antibody or antigen binding fragment thereof, a cytokine, a growth factor, a growth factor receptor, an interferon, an interleukin, an inflammatory molecule, an oncogene product, a peptide hormone, a signal transduction molecule, a steroid hormone receptor, a transcriptional modulator protein (e.g., a transcriptional activator protein (such as GAL4), or a transcriptional repressor protein, etc.) or a portion thereof. In certain embodiments, the protein of interest comprises a therapeutic protein, a diagnostic protein, an industrial enzyme, or a portion(s) thereof.

In an embodiment, the protein of interest that is produced by the methods described herein are further modified through the one or more nsAA(s). For example, the nsAA can be modified through, e.g., a nucleophilic-electrophilic reaction, through a [3+2]cycloaddition, etc. In certain embodiments, the protein produced by the methods described herein is modified by at least one post-translational modification (e.g., N-glycosylation, O-glycosylation, acetylation, acylation, lipid-modification, palmitoylation, palmitate addition, phosphorylation, glycolipid-linkage modification, and the like) in vivo.

In an aspect, the protein or polypeptide of interest (or portion thereof) is encoded by a nucleic acid. Typically, the nucleic acid comprises at least one selector codon, at least two selector codons, at least three selector codons, at least four selector codons, at least five selector codons, at least six selector codons, at least seven selector codons, at least eight selector codons, at least nine selector codons, or even ten or more selector codons.

Kits

Kits are also a feature of this disclosure. For example, a kit for producing a protein that comprises at least one nsAA is provided. In certain embodiments, the kit includes an O-tRNA or a polynucleotide sequence coding for an O-tRNA or comprising a O-tRNA, and/or an O—RS or a polynucleotide sequence encoding an O-RS. In certain embodiments, the kit includes cells comprising a polynucleotide sequence comprising an O-tRNA, and/or an O—RS or a polynucleotide sequence encoding an O-RS. In certain embodiments, the cells comprise a polynucleotide encoding a polypeptide or protein of interest. In certain embodiments, the kit further includes at least one nsAA. In certain embodiments, the kit further comprises instructional materials for producing the protein.

Methods for Incorporating nsAA into Polypeptides at Specific Locations

The present invention also provides methods for producing at least one protein in a prokaryotic (e.g., Eubacteria) or eukaryotic (e.g., yeast, protist mammalian, plant, or insect) translation system. In certain embodiments, a cell, e.g., an E. coli cell, comprising the tRNA of the present invention includes such a translation system. The translation system is provided with the at least one non-standard amino acid, thereby producing at least one protein containing at least one non-standard amino acid. The compositions and methods described here can be used with non-standard amino acids, e.g., providing specific spectroscopic, chemical, or structural properties to proteins using any of a wide array of side chains. These compositions and methods are useful for the site-specific incorporation of non-standard amino acids via selector codons, e.g., stop codons, four base codons, and the like. The translation system is also provided with an orthogonal tRNA (O-tRNA), that functions in the translation system and recognizes the at least one selector codon and an orthogonal aminoacyl tRNA synthetase (0-RS), that preferentially aminoacylates the O-tRNA with the at least one non-standard amino acid in the translation system.

The disclosure also relates to a method of producing a polypeptide comprising at least one nsAA, comprising expressing in a cell an O-tRNA and an O-RS as described herein. In certain embodiments, the orthogonal tRNA synthetase (O-RS) comprises a substitution of at least one of the following residues as compared to a wild-type M. jannaschii tRNA synthetase (SEQ ID NO:45): (a) T11, (b) I15, (c) D27, (d) M96, (e) G97, and (f) K101R. In certain embodiments, the orthogonal tRNA synthetase comprises at least 85% sequence identity to SEQ ID NO: 45 but does not comprise SEQ ID NO: 35. In certain embodiments, the O-RS further comprises a substitution at D286. In certain embodiments, the substitution at D286 is a D286R substitution. In certain embodiments, the O-RS comprises at least one of the following substitutions: (a) T11A, T11V, T11I, T11L, T11G; (b) I15V, I15A, I15L, I15G; (c) D27G, D27A, D27V, D27I, D27L; (d) M96I, M96A, M96V, M96L, M96G; (e) G97D, G97E; or (f) K101R, K101H, and K101K. In certain embodiments, the O-RS comprises at least one of the following substitutions: (a) TI lA, (b) I15V, (c) D27G, (d) M96I, (e) G97D, and (f) K101R.

In certain embodiments, the O-RS comprises at least one of the following substitutions: (a) T11A, T11V, T11I, T11L, or T11G; (b) D27G, D27A, D27V, D27I, or D27L; (c) H45Y, H45W, or H45F; (d) M96I, M96A, M96V, M96L, or M96G; (e) G97D or G97E; (f) K101R, K101H, or K101K; (g) G158D or G158E (h) E135K, E135R or E135H; and (i) S269G, S269A, or S269C.

In certain embodiments, the O-RS comprises an additional substitution of at least one of the following residues as compared to a wild-type M. jannaschii tRNA synthetase (SEQ ID NO:45): 257, 261 and 284. In certain embodiments, the O-RS comprises at least one of the following substitutions: R257W, R257F, R257Y, R257H, F261P, E272V, P284S, P284A, P284G, P284C, P284V, M285D, M285F, R286V, and R286Y.

In certain embodiments, the O-RS comprises the mutations: I15V, D286R, T11A, D27G, M96I, G97D, K101R, R257W, and P284S.

In certain embodiments, the O-RS comprises the mutations: I15V, D286R, T11A, D27G, M96I, G97D, K101R, R257W, and F261P.

In certain embodiments, the O-RS comprises the mutations: I15V, D286R, T11A, D27G, M96I, G97D, K101R, R257W, F261P and P284S.

In certain embodiments, the O-RS comprises the mutations: I15V, T11A, D27G, M96I, G97D, K101R, R257W, F261P and D286Y.

In certain embodiments, the O-RS comprises the mutations: I15V, T11A, D27G, M96I, G97D, K101R, and D286Y.

In certain embodiments, the O-RS comprises the mutations: I15V, T11A, D27G, M96I, G97D, K101R, F261P and D286Y.

In certain embodiments, the O-RS further comprises one or more substitutions selected from K90Q, I176L, R257W, P258A, F261P, E272V, H283L, H283T, P284V, P284S, M285F, M285D, D286Y, and D286V.

In certain embodiments, the O-RS comprises the mutations: I15V, D286R, T11A, D27G, M96I, G97D, K101R, and M285D.

In certain embodiments, the O-RS comprises the mutations: I15V, D286R, T11A, D27G, M96I, G97D, K101R, and M285F.

In certain embodiments, the O-RS comprises the mutations: I15V, T11A, D27G, M96I, G97D, K101R, and R286Y.

In certain embodiments, the O-RS comprises the mutations: I15V, T11A, D27G, M96I, G97D, K101R, and R286V.

In certain embodiments, the O-RS comprises the mutations: I15V, T11A, D27G, M96I, G97D, K101R, and R257W.

In certain embodiments, the O-RS comprises the mutations: I15V, T11A, D27G, M96I, G97D, K101R, and P284S.

In certain embodiments, the O-RS comprises the mutations: I15V, T11A, D27G, M96I, G97D, K101R, and F261P.

In certain embodiments, the O-RS comprises the mutations: I15V, T11A, D27G, M96I, G97D, K101R, and E272V.

In certain embodiments, the O-RS comprises an amino acid sequence that is at least 85% identical to a sequence selected from SEQ ID NOs: 39-44 and 46-56. In certain embodiments, the amino acid sequence is at least 90% identical, at least 91% identical, at least 92% identical, at least 93% identical, at least 94% identical, at least 95% identical, at least 96% identical, at least 97% identical, at least 98% identical, or at least 99% identical to a sequence selected from SEQ ID NOs: 39-44 and 46-56. In certain embodiments, the amino acid sequence is selected from SEQ ID NOs: 39-44 and 46-56.

In certain embodiments, the O-tRNA comprises a nucleic acid sequence set forth in SEQ ID NO: 2.

In certain aspects, described herein are methods for producing a polypeptide comprising at least one nsAA, comprising providing: i) an O-tRNA comprising a nucleic acid sequence set forth in SEQ ID NO: 2; and wherein the O-tRNA is capable of being aminoacylated with at least one non-standard amino acid (nsAA) by an O-RS; ii) an O-RS; wherein the O-RS aminoacylates the O-tRNA with the nsAA; and iii) a polynucleotide encoding the polypeptide, wherein the polynucleotide comprises at least one selector codon; and wherein the O-tRNA recognizes the selector codon.

In certain embodiments, the method further comprises expressing an O-RS described herein in the cell.

In certain embodiments, the protein or polypeptide of interest (or portion thereof) is comprises at least one nsAA, at least two nsAAs, at least three nsAAs, at least four nsAAs, at least five nsAAs, at least six nsAAs, at least seven nsAAs, at least eight nsAAs, at least nine nsAAs, or even ten or more nsAAs.

This disclosure also provides methods for producing, in a cell, at least one protein of interest comprising at least one nsAA. The methods include, e.g., growing, in an appropriate medium, a cell that comprises a nucleic acid that comprises at least one selector codon and encodes the protein.

In an embodiment, the method further includes incorporating into the protein of interest the nsAA, where the nsAA comprises a first reactive group; and contacting the protein with a molecule (e.g., a dye, a polymer, e.g., a derivative of polyethylene glycol, a photocrosslinker, a cytotoxic compound, an affinity label, a derivative of biotin, a resin, a second protein or polypeptide, a metal chelator, a cofactor, a fatty acid, a carbohydrate, a polynucleotide (e.g., DNA, RNA, etc.), etc.) that comprises a second reactive group. The first reactive group reacts with the second reactive group to attach the molecule to the nsAA through a [3+2] cycloaddition. In an embodiment, the first reactive group is an alkynyl or azido moiety and the second reactive group is an azido or alkynyl moiety. For example, the first reactive group is the alkynyl moiety (e.g., in nsAA p-propargyloxyphenylalanine) and the second reactive group is the azido moiety. In another example, the first reactive group is the azido moiety (e.g., in the nsAA p-azido-L-phenylalanine) and the second reactive group is the alkynyl moiety.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Selection Scheme for the Directed Evolution of O-RS

In this Example, incorporation of nsAAs into proteins utilizes three components: 1) a suitable nsAA, 2) a tRNA molecule that is orthogonal to native tRNA molecules (i.e., the tRNA is not readily aminoacylated by native tRNA synthetases in the cell) which has an engineered anticodon to recognize the amber codon, and 3) an orthogonal aminoacyl tRNA synthetase that can aminoacylate the nsAA onto the orthogonal tRNA (and that will not readily aminoacylate the nsAA onto native tRNAs). The following sections describe the identification of a suitable system for robust nsAA incorporation at amber codons (TAG) in E. coli SoluPro®.

Candidate amino acids and orthogonal tRNA/aminoacyl tRNA synthetase pairs were selected to serve as a starting point for additional engineering:

Orthogonal tRNA/Aminoacyl tRNA Synthetase Pair

The M. jannaschii tyrosyl-tRNA synthetase (MjtRNA^{Tyr}_{CUA}/MjYRS) pair has been the most extensively used starting point for the evolution of Orthogonal Translation Systems (OTSs) that incorporate non-standard amino acids in E. coli. The MjYRS does not aminoacylate any endogenous E. coli tRNAs with tyrosine, but aminoacylates a mutant tyrosine amber suppressor (mutRNA_{CUA}). By applying a combination of synergized approaches, such as high-throughput cloning, FACS sorting, NGS analysis, in vitro evolution, synthetic biology, structural biology, and artificial intelligence, a series of variants for MjtRNA^{TYr}_{CUA}/MjYRS pairs with significantly enhanced activity for the incorporation of non-standard amino acids into proteins were identified. Candidate MjtRNA^{Tyr}_{CUA}/MjYRS pairs were evaluated for incorporation efficiency in Example 2.

### Example 2: Evaluation of the Incorporation Efficiency of Candidate OTSs

The performance of orthogonal translation systems (OTSs) varies greatly in terms of the efficiency and accuracy of nsAA incorporation. To enable rapid and systematic comparisons of these critical parameters, a toolkit for characterizing any Escherichia co/i OTS that reassigns the amber stop codon (TAG) was used. It assesses OTS performance by measuring the efficiency, i.e., relative readthrough efficiency (RRE) and fidelity, i.e., maximum misincorporation frequency (MMF), in the presence and absence of the nsAA of interest. The relative readthrough efficiency (RRE) of the TAG codon is the GFP/RFP fluorescence ratio for the mcherryTAG assay plasmid divided by the GFP/RFP fluorescence ratio for the mcherryTAC control plasmid. For an efficient aaRS-tRNA pair, the RRE when the nsAA is present in the media should approach or surpass a value of one, as this metric reflects how well the TAG amber codon is translated compared to the TAC tyrosine codon. The fidelity of an OTS was evaluated by comparing the RRE values obtained with and without nsAA present. Specifically, an (in) fidelity metric, the maximum misincorporation frequency (MMF), is calculated by dividing the RRE when nsAA is not added to the growth media by the RRE when nsAA is present. An ideal OTS has an MMF value of zero, reflecting that no GFP is produced unless the nsAA is present. Note, however, that MMF is a very strict measure of fidelity. Some engineered aaRS tRNA pairs are known to incorporate mostly the nsAA when it is provided at a sufficiently high concentration, but to nonspecifically aminoacylate the tRNA with a standard amino acid instead when it is more abundant.

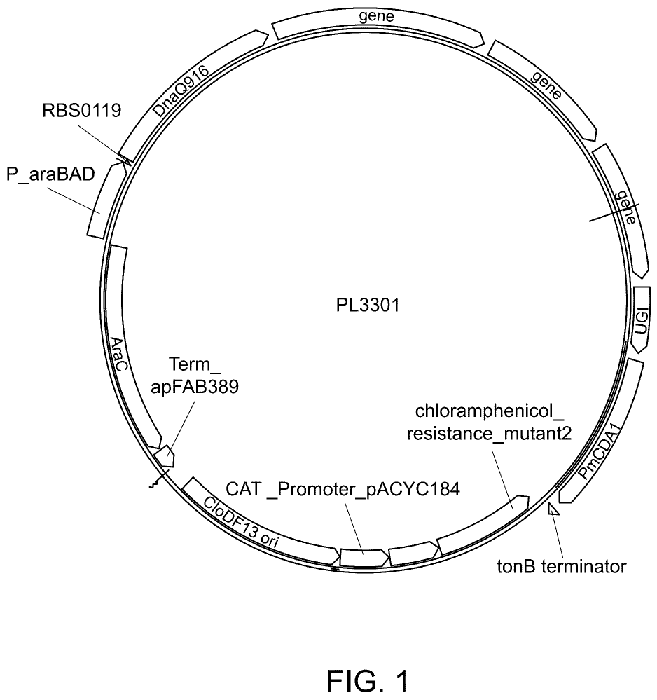

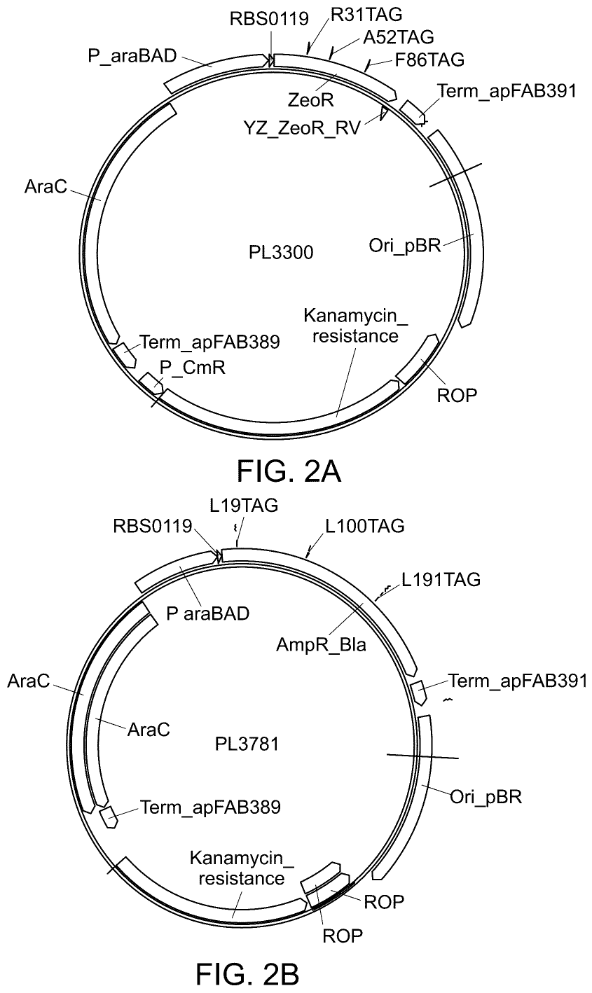

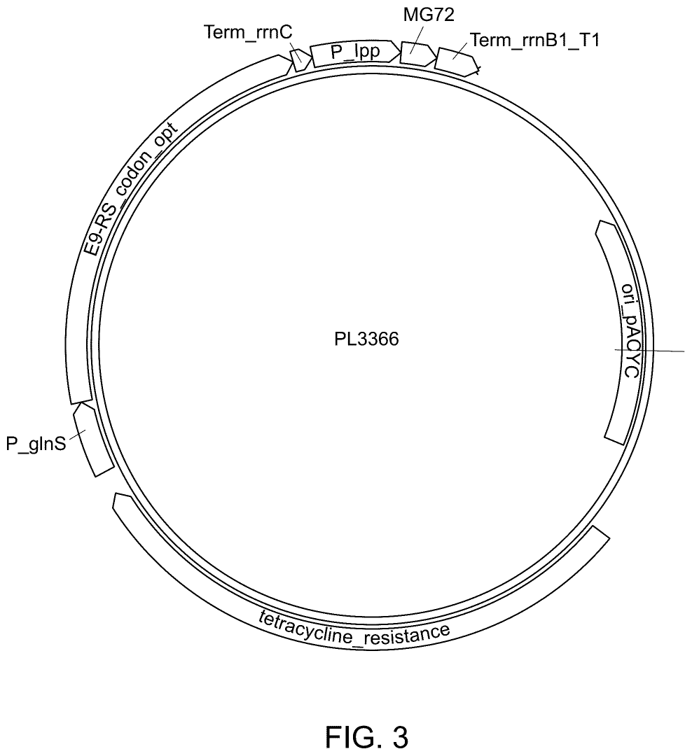

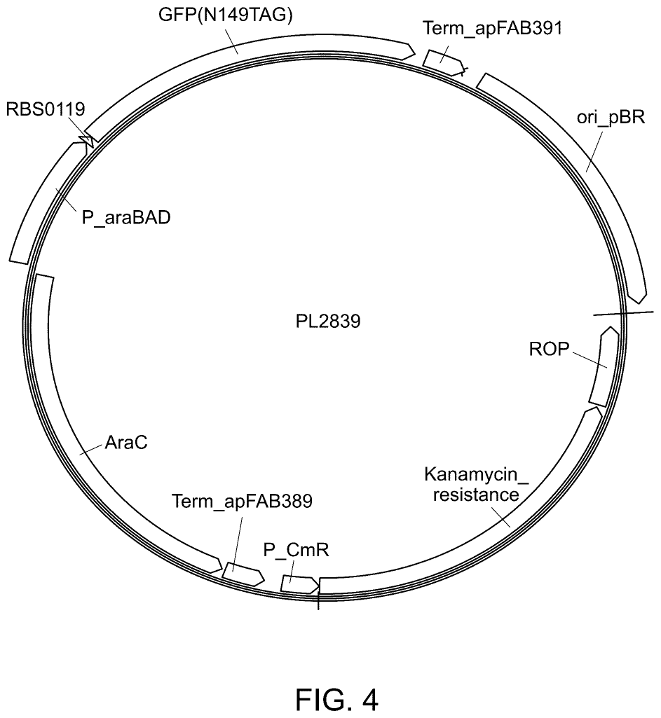

To examine the utility of the engineered OTSs for incorporating nsAA into polypeptides, the constructs harboring candidate variants of MjtRNA^{Tyr}_{CUA}/MjYRS pairs (FIG. 3) were transformed with a wtGFP expressing construct (FIG. 4). As shown in FIG. 1, the constructs containing candidate OTSs contained a low-copy number origin of replication pACYC and a tetracycline resistance cassette. Expression of O-tRNA MG72 (SEQ ID NO: 2) was driven by the constitutive promoter plpp and terminated by rmB1 terminator while the expression of O-RS was controlled by the constitutive promoter pgln and terminated by rmC1 terminator. The N149 codon of wtGFP was mutated to an amber codon.

Cells were cultured in LB (10 g/L tryptone, 5 g/L yeast extract, 10 g/L NaCl). Tetracycline (15 μg/mL), kanamycin (50 μg/mL), arabinose (250 μM), propionate (20 mM) were added as appropriate. Amino acids 4-acetyl-L-phenylalanine (A206865), 4-propargyloxy-L-phenylalanine (A721556), 4-Amino-L-phenylalanine hydrate (A943099) were purchased from Ambeed. 4-azido-L-phenylalanine (909564) was purchased from Sigma-Aldrich. Stock solutions of L-tyrosine was prepared at 500 mM in dH2O. For 4-acetyl-L-phenylalanine, 4-propargyloxy-L-phenylalanine, 4-Amino-L-phenylalanine hydrate and 4-azido-L-phenylalanine, a 500 mM stock solution was prepared in in 1 M NaOH solution. All amino acid stocks were sterilized using 0.22 μm filters. All stock solutions of nsAA were prepared as 500×.

Each OTS system was constructed by using Golden Gate Assembly (New England Biolabs) to clone the respective O-tRNA mutants into plasmids. Each OTS plasmid was transformed separately into E. coli strain EB114 which already contained assay plasmid and that were made electrocompetent by 10% glycerol washes. The aaRS and tRNA genes in these clones were sequenced using Illumina technology to verify that no mutations had occurred in the OTS cassette prior to testing them using the nsAA incorporation measurement kit.

For kit assays, strains were revived from −80° C. glycerol stocks in 10 mL LB in 50 mL Erlenmeyer flasks with kanamycin and tetracycline. These cultures were incubated at 37° C. with orbital shaking over a 1-inch diameter at RPM for 24 h. From these preconditioned cultures, a diluted culture was prepared by concentrating cells in 500 μL of culture by centrifugation at 4° C., decanting the supernatant, and then adding 10 mL of fresh media with antibiotics, arabinose and +/−nsAA. This procedure creates an overall 1:100 dilution in fresh media. Cultures lacking nsAA were supplemented with an equivalent amount of sterile dH2O water to achieve a consistent LB concentration. Sample blanks+/−nsAA were prepared in an identical way, but omitting cells.

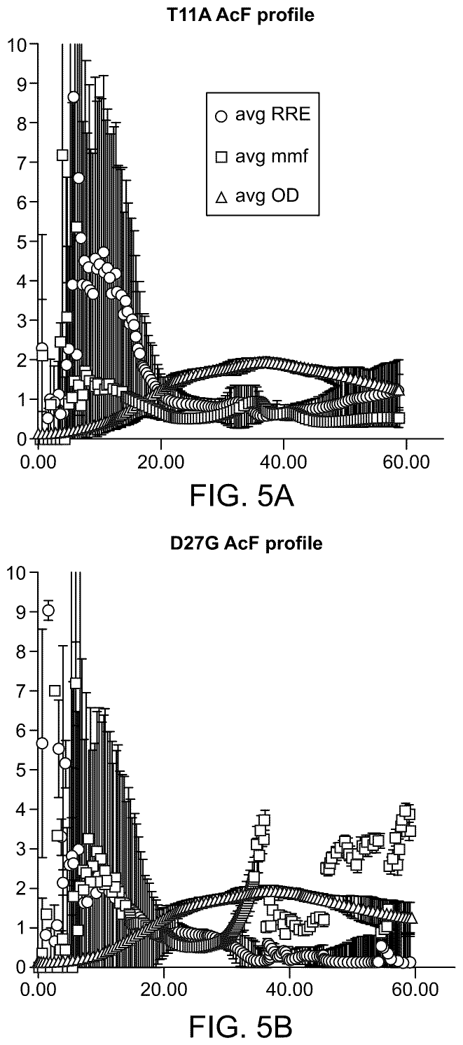

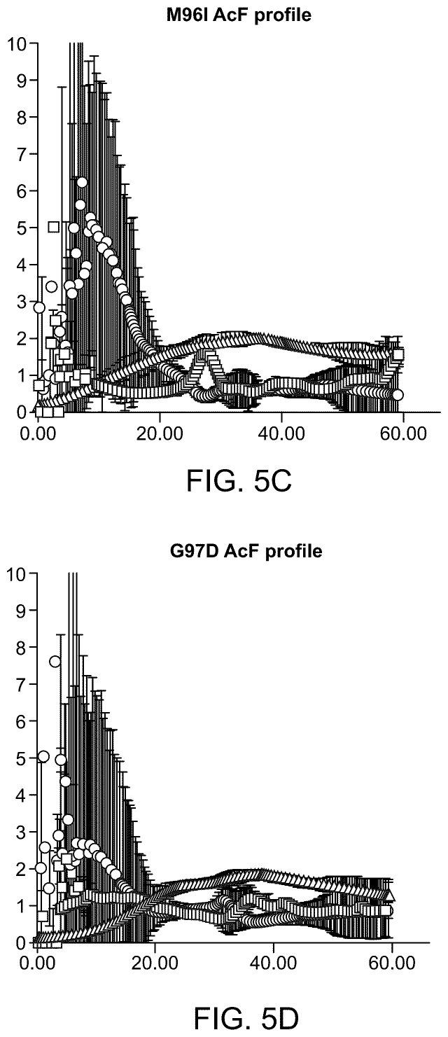

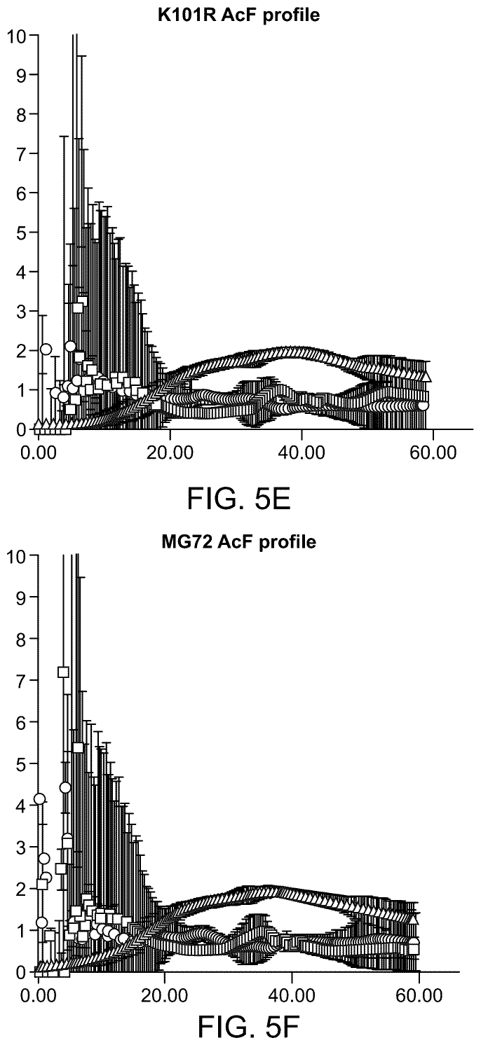

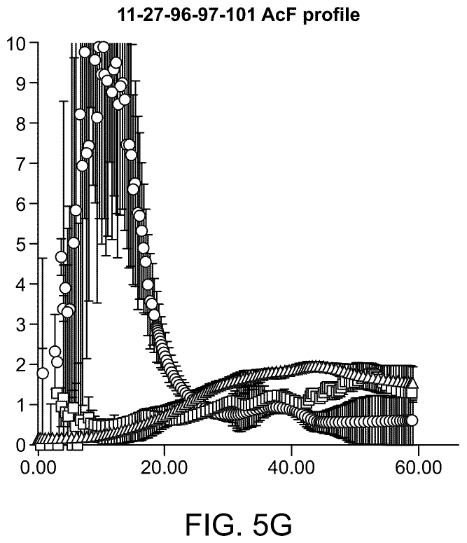

Fluorescence and OD readings were made in using an Infinite Enspire microplate reader (PerkinElmer). To test the candidate OTSs, four biological replicates were compared. For each experiment, a Costar #3631 black clear-bottom 96-well plate was filled with 150 μL aliquots of each sample and blank to be tested. The assay was run for 30 h in the microplate reader while it was incubated continuously at 30° C. and shaken 15 s before and after readings. OD, and GFP measurements were taken every 20 min. OD was measured at 600 nm (OD600). The GFP excitation was set at 395 nm with emission at 509 nm. The OD, RRE and MMF values determined for each of these sets of four wells at each time point were then averaged over this time window to create summary scores for that replicate. FIGS. 5A-5G provide OD, RRE, and MMF summary scores for an OTS comprising a T11A mutant (FIG. 5A), a D27G mutant (FIG. 5B), a M96I mutant (FIG. 5C), a G97D mutant (FIG. 5D), a K101R mutant (FIG. 5E), a control using an MG72 O-tRNA (FIG. 5F), and a T11A; D27G; M96I; G97D, K101R mutant (FIG. 5G).

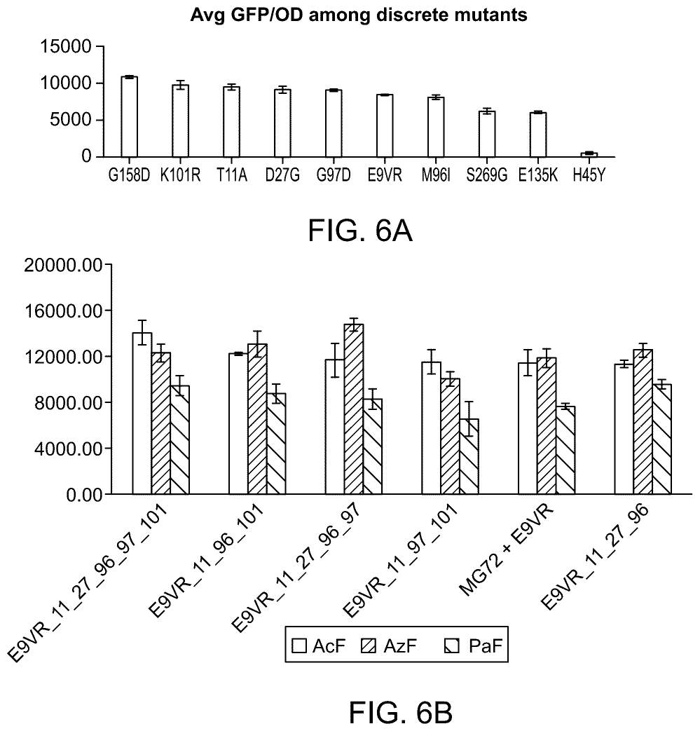

In addition, OD normalized GFP intensity scores for various mutants are shown in FIG. 6A and FIG. 6B. As shown in FIG. 6A, mutating the candidate O-RS at G158D, K101R, T11A, D27G, G97D, and M96I resulted in an O-RS having superior summary scores (OD normalized GFP intensity shown on Y-axis) as compared to the E9VR mutant. FIG. 6B shows the summary scores (OD normalized GFP intensity) for multiply mutant O-RS as compared to the E9VR mutant (using the MG72 tRNA) using three different nsAAs (AcF, AzF, and PaF).

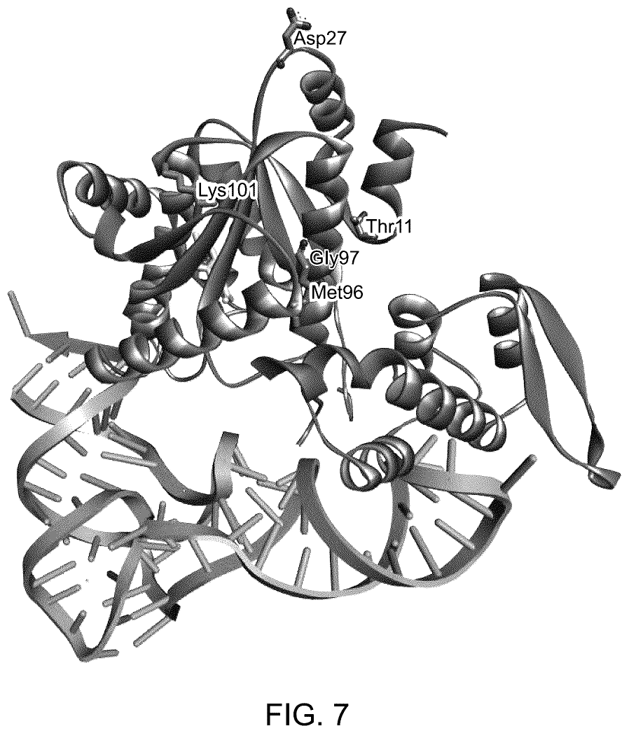

FIG. 7 provides a schematic representation of an O-RS complexed with a tRNA, in which the positions of the K101, T11, D27, G97, and M96 residues are shown.

### Example 3: Screening of Additional Mutations for Further Improvement of Incorporation Efficiency of Candidate OTSs

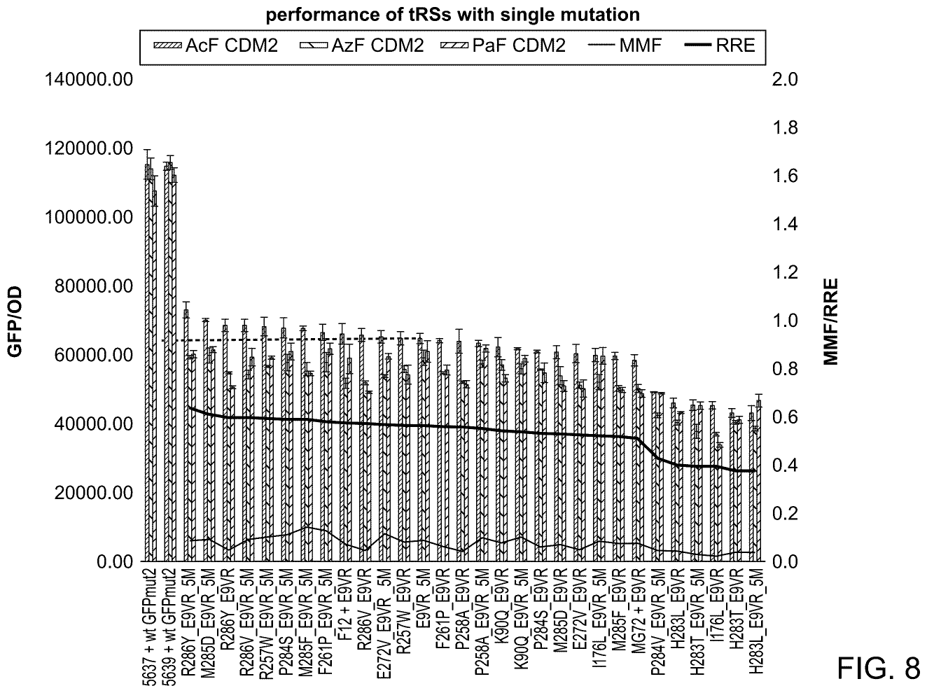

The starting point for O-RS directed evolution was E9VR, which contains two mutations, I15V and D286R. A variant identified for having improved AcF incorporation from the permutation pool of single mutations was an O-RS comprising the mutations: E9VR, T11A, D27G, M96I, G97D and K101R, which was named E9VR_5mut E9VR and E9VR_5mut were selected as the starting point for the new round of directed evolution to assess the effect of the following additional substitutions on the O-RS: K90Q, I176L, R257W, P258A, F261P, E272V, H283L, H283T, P284V, P284S, M285F, M285D, D286Y, and D286V. E9VR and E9VR_5mut were chosen as the starting points and the single mutations were introduced to evaluate their utility in the expression system SoluPro™. Because the activities of these O-RS were expected to reach high levels, an additional TAG codon in the reporter GFP protein was introduced to elevate the challenge for the orthogonal translation system (OTS). Testing the ability of the OTS to read through two consecutive amber codons accentuated the gain in the new round of directed evolution. The performance of these variants with single mutations depicted as OD normalized fluorescence in presence of AcF, AzF and PaF respectively are shown in FIG. 8. Wild type GFP was used as the positive control, and fluorescence measured in absence of nsAA was used as the negative control. Relative read-through efficiency (RRE) and maximum misincorporation frequency (MMF) were calculated and are presented in the overlaid line graph. The dashed line highlighted the performance gain relative to E9VR_5mut.

E9VR_5mut clearly stood out as a better starting point of evolution compared to E9VR, confirming the utility of the previous engineering effort. Based on p-values, some variants clearly stood out as advantageous mutations (as shown in Table below). Identified single mutations on O-RS improved the performance by 5-10% from starting point E9VR_5mut.

| Variants | P- Value |
|---|---|
| F261P | 0.0075 |
| D286V | 0.0102 |
| D286Y | 0.0102 |
| M285D | 0.0147 |
| R257W | 0.0221 |
| M285D_5M | 0.0235 |
| M285F_5M | 0.0286 |
| M285F | 0.0299 |
| D286Y_5M | 0.0336 |
| D286V_5M | 0.0510 |
| R257W_5M | 0.0523 |
| P284S_5M | 0.0547 |
| F261P_5M | 0.0584 |
| P284S | 0.0712 |
| E272V | 0.1286 |
| E272V_5M | 0.2763 |

### Example 4: Stacking of Privileged Single Mutations of O-RS Results in Improved nsAA Incorporation Performance

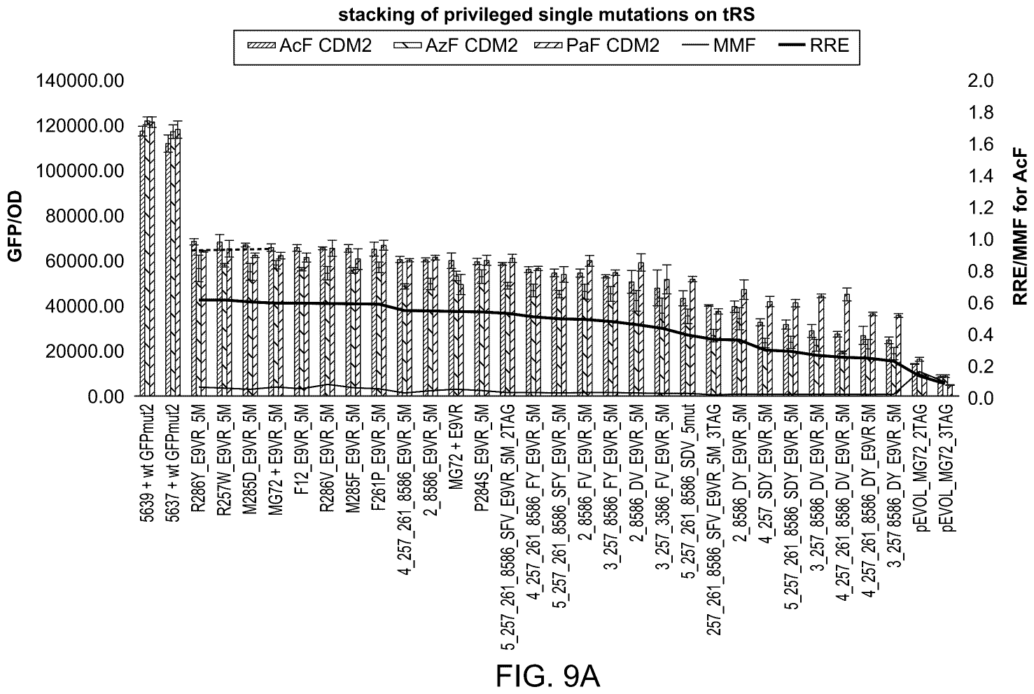

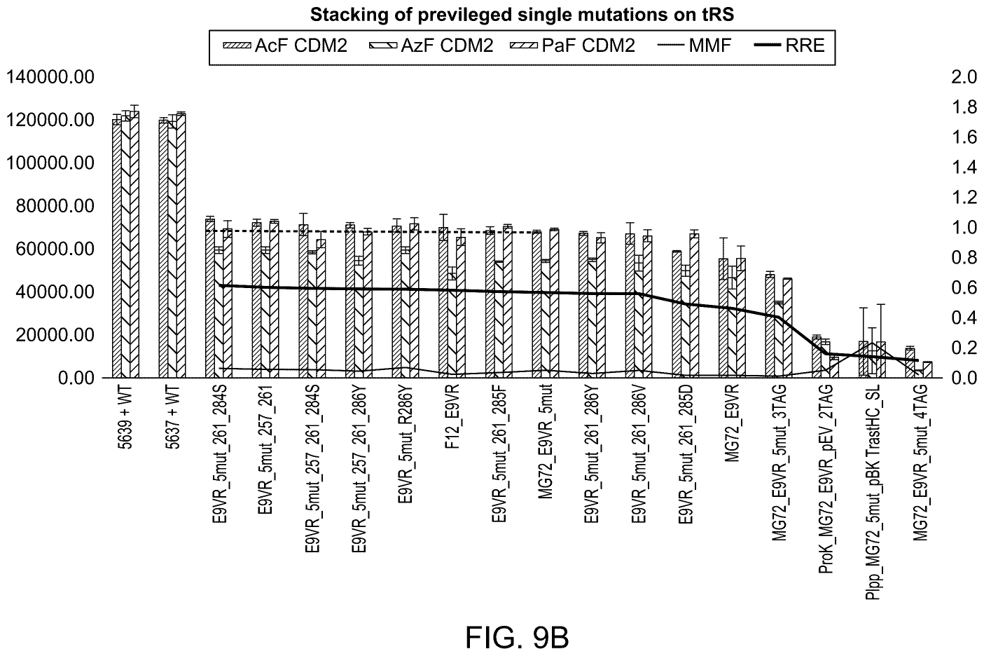

From the results of Example 3, eight single mutations were selected for stacking: D286Y, M285D, D286V, R257W, P284S, M285F, F261P, E272V, to identify combinations of mutations that will yield further increased nsAA incorporation performance. As shown in FIG. 9, mutants E9VR_5mut_R257W_P284S, E9VR_5mut_R257W_F261P, E9VR_5mut_R257W_F261P_P284S, E9VR_5mut R257W F261P D286Y, E9VR_5mut D286Y and E9VR_5mut_F261P_D286Y, stood out as best performers from the permutation pool in terms of both RRE and MMF. Up to 9% performance improvement was achieved through the stacking of privileged single mutation relative to E9VR_5mut. E9VR_5mut_D286Y was selected and renamed E9VR_6mut for additional analysis of OTS/plasmid backbone permutations.

### Example 5: OTS/Backbone/tRNA Promoter Permutation and Additional Single Mutation Stacking Yielded Various Highly Efficient OTS with Incorporation Performance Improvement

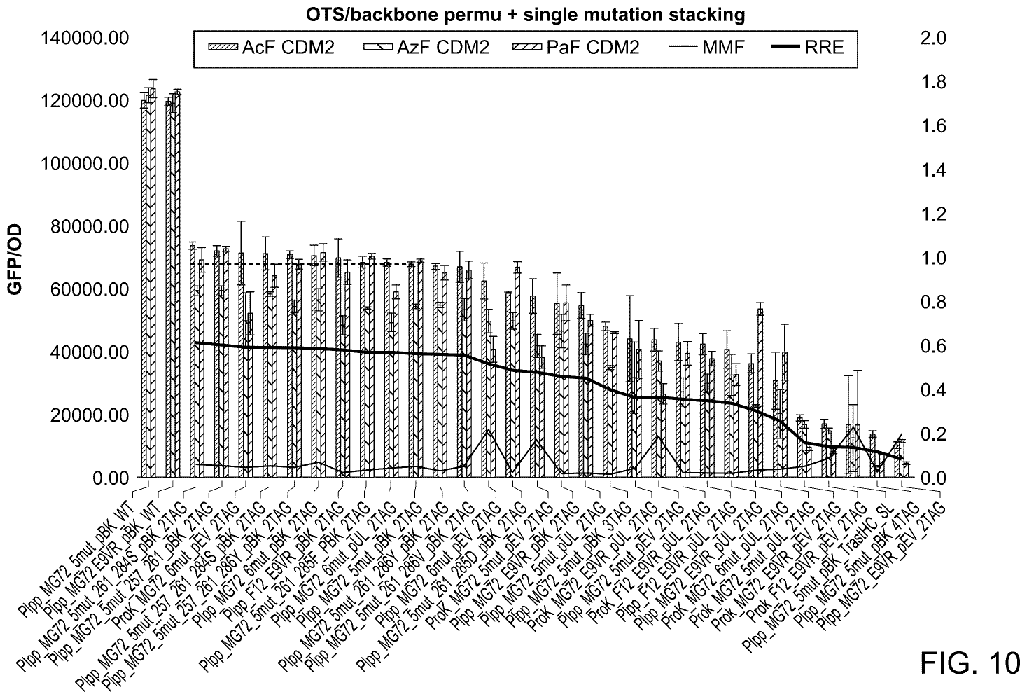

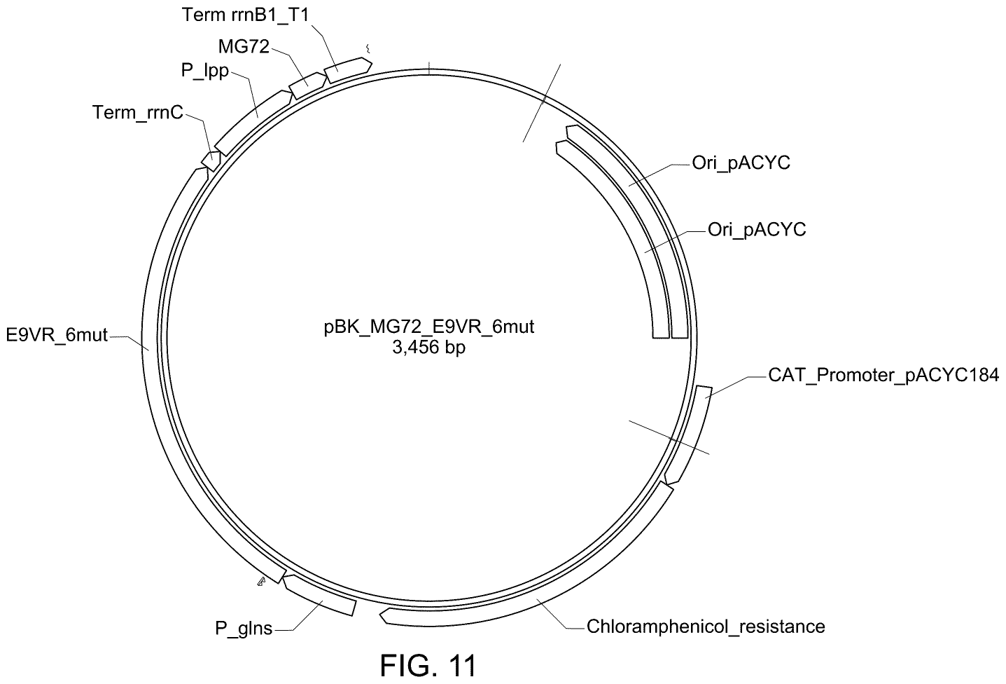

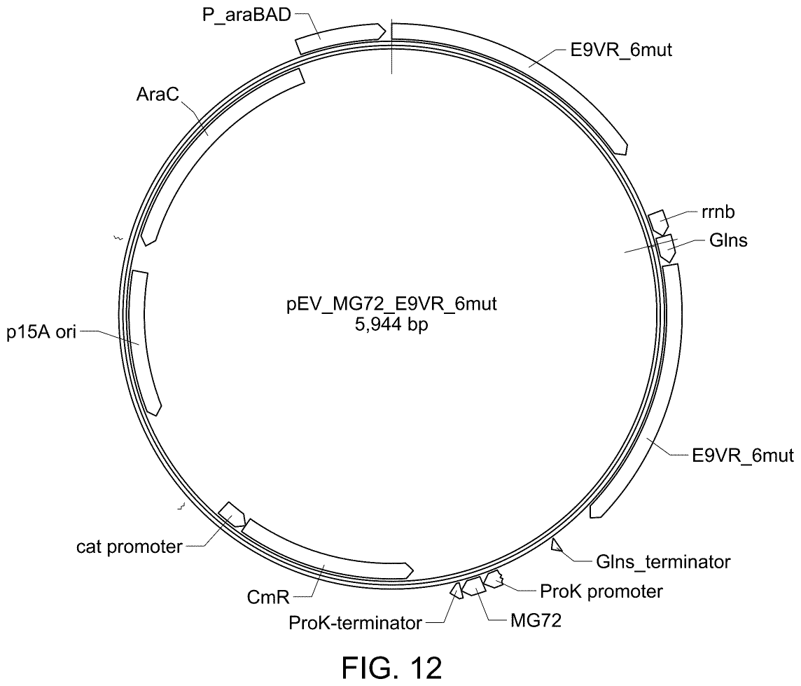

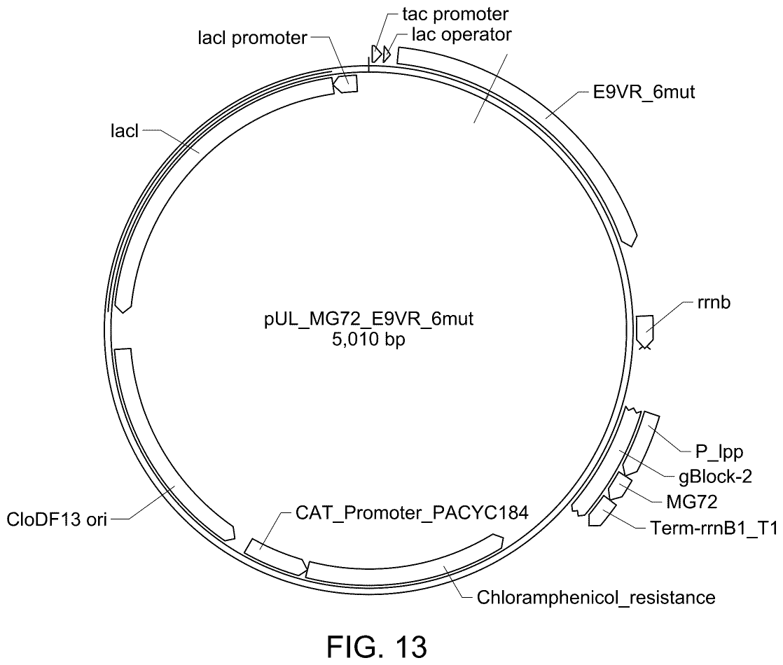

Four OTSs (F12_E9VR, MG72_E9VR, MG72_E9VR_5mut and MG72_E9VR 6mut), two promoters for tRNA (plpp and proK), and three plasmid backbones (pBK, pEV and pUL) were permuted and screened for nsAA incorporation efficiency as conducted in Examples 3 and 4, using GFP with 2 amber codons. FIGS. 11-13 are diagrams depicting examples of the three plasmid backbones used. FIG. 10 shows the results of the screening of the permuted constructs. The dashed line highlighted the performance gain relative to E9VR_5mut in the pBK backbone.

For each of the plasmid backbones, at least one OTS/Backbone combination was identified that surpassed the performance of E9VR_5mut in the pBK backbone. More specifically, Plpp_MG72_E9VR_6mut_pBK, ProK_MG7, E9VR_6mut_pEV and Plpp_E9VR_MG72_6mut_pUL demonstrated up to 9% increase in RRE without sacrificing the fidelity of the incorporation (MMF). OTS MG72_E9VR_6mut generally outperformed the other three tested OTS, confirming the utility of the benefit of previous directed evolution. Promoter plpp was beneficial for backbone pBK and pUL; however, promoter ProK was the best promoter for the tRNA in the backbone pEV.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

SEQUENCE LISTING

| SEQ ID | | |
|---|---|---|
| NO | DESCRIPTION | SEQUENCE |
| SEQ ID | F12 O-tRNA | CCGGCGGUAGUUCAGCAGGGCAGAACGGCGGACUCUAAA |
| NO: 1 | sequence | UCCGCAUGGCGCCGGUUCAAAUCCGGCCCGCCGGACCA |
| SEQ ID | MG72 O-tRNA | CCGGCGGUAGUUCAGAGGGCAGAACGGCGGACUCUAAAU |
| NO: 2 | sequence | CCGCAUGGCGCCAGUUCAAAUCUGGCCCGCCGGACCA |
| SEQ ID | MG24 O-tRNA | CCCGCCUUAGUUCAGAGGGCAGAACGGCGGACUCUAAAUC |
| NO: 3 | sequence | CGCAUGGCGCCGGUUCAAAUCCGGCAGGCGGGACCA |
| SEQ ID | MG33 O-tRNA | CCGGCGGUAGUUCAGAGGGCAGAACGGCGGACUCUAAAU |
| NO: 4 | sequence | CCGCAUGGCGCCGGUUCAAAUCCGGCCCGCCGGACCA |
| SEQ ID | MG16 O-tRNA | CCCGCCUUAGUUCAGAGGGCAGAACGGCGGACUCUAAAUC |
| NO: 5 | sequence | CGCAUGGCACGGGUUCAAAUCCCGUAGGCGGGACCA |
| SEQ ID | MG17 O-tRNA | CCGGCGGUAGUUCAGAGGGCAGAACGGCGGACUCUAAAU |
| NO: 6 | sequence | CCGCAUGGCAGGGGUUCAAAUCCCCUCCGCCGGACCA |
| SEQ ID | MG30 O-tRNA | CCCGCCUUAGUUCAGCAGGGCAGAACGGCGGACUCUAAAU |
| NO: 7 | sequence | CCGCAGGUCGCCGGUUCAAAUCCGGCAGGCGGGACCA |
| SEQ ID | MG36 O-tRNA | CCCGCCUUAGUUCAGAGGGCAGAACGGCGGACUCUAAAUC |
| NO: 8 | sequence | CGCAGGUCGCCGGUUCAAAUCCGGCAGGCGGGACCA |
| SEQ ID | MG22 O-tRNA | CCCGCCUUAGUUCAGCAGGGCAGAACGGCGGACUCUAAAU |
| NO: 9 | sequence | CCGCAGGUCACGGGUUCAAAUCCCGUAGGCGGGACCA |
| SEQ ID | MG25 O-tRNA | CCGGCGGUAGUUCAGAGGGCAGAACGGCGGACUCUAAAU |
| NO: 10 | sequence | CCGCAUGGCACGGGUUCAAAUCCCGUCCGCCGGACCA |
| SEQ ID | MG29 O-tRNA | CCGGCGGUAGUUCAGAGGGCAGAACGGCGGACUCUAAAU |
| NO: 11 | sequence | CCGCAGGUCAGGGGUUCAAAUCCCCUCCGCCGGACCA |
| SEQ ID | MG35 O-tRNA | CCGGCGGUAGUUCAGCAGGGCAGAACGGCGGACUCUAAA |
| NO: 12 | sequence | UCCGCAUGGCAGGGGUUCAAAUCCCCUCCGCCGGACCA |
| SEQ ID | MG37 O-tRNA | CCGGCGGUAGUUCAGAGGGCAGAACGGCGGACUCUAAAU |
| NO: 13 | sequence | CCGCAGGUCACGGGUUCAAAUCCCGUCCGCCGGACCA |
| SEQ ID | MG62 O-tRNA | CCGGAGGUAGUUCAGCAGGGCAGAACGGCGGACUCUAAA |
| NO: 14 | sequence | UCCGCAUGGCGCCGGUUCAAAUCCGGCCCUUCGGACCA |
| SEQ ID | MG97 O-tRNA | CCGGUGUUAGUUCAGAGGGCAGAACGGCGGACUCUAAAU |
| NO: 15 | sequence | CCGCAUGUCACGGGUUCAAAUCCCGUACACCGGACCA |
| SEQ ID | MG109 O- | CCGACCUUAGUUCAGAGGGCAGAACGGCGGACUCUAAAU |
| NO: 16 | tRNA sequence | CCGCAUGUCAGGGGUUCAAAUCCCCUAGGUCGGACCA |
| SEQ ID | MG72 O-tRNA | CCGGCGGTAGTTCAGAGGGCAGAACGGCGGACTCTAAATC |
| NO: 17 | DNA sequence | CGCATGGCGCCAGTTCAAATCTGGCCCGCCGGACCA |
| SEQ ID | MG24 O-tRNA | CCCGCCTTAGTTCAGAGGGCAGAACGGCGGACTCTAAATCC |
| NO: 18 | DNA sequence | GCATGGCGCCGGTTCAAATCCGGCAGGCGGGACCA |
| SEQ ID | MG33 O-tRNA | CCGGCGGTAGTTCAGAGGGCAGAACGGCGGACTCTAAATC |
| NO: 19 | DNA sequence | CGCATGGCGCCGGTTCAAATCCGGCCCGCCGGACCA |
| SEQ ID | MG16 O-tRNA | CCCGCCTTAGTTCAGAGGGCAGAACGGCGGACTCTAAATCC |
| NO: 20 | DNA sequence | GCATGGCACGGGTTCAAATCCCGTAGGCGGGACCA |
| SEQ ID | MG17 O-tRNA | CCGGCGGTAGTTCAGAGGGCAGAACGGCGGACTCTAAATC |
| NO: 21 | DNA sequence | CGCATGGCAGGGGTTCAAATCCCCTCCGCCGGACCA |
| SEQ ID | MG30 O-tRNA | CCCGCCTTAGTTCAGCAGGGCAGAACGGCGGACTCTAAATC |
| NO: 22 | DNA sequence | CGCAGGTCGCCGGTTCAAATCCGGCAGGCGGGACCA |
| SEQ ID | MG36 O-tRNA | CCCGCCTTAGTTCAGAGGGCAGAACGGCGGACTCTAAATCC |
| NO: 23 | DNA sequence | GCAGGTCGCCGGTTCAAATCCGGCAGGCGGGACCA |
| SEQ ID | MG22 O-tRNA | CCCGCCTTAGTTCAGCAGGGCAGAACGGCGGACTCTAAATC |
| NO: 24 | DNA sequence | CGCAGGTCACGGGTTCAAATCCCGTAGGCGGGACCA |
| SEQ ID | MG25 O-tRNA | CCGGCGGTAGTTCAGAGGGCAGAACGGCGGACTCTAAATC |
| NO: 25 | DNA sequence | CGCATGGCACGGGTTCAAATCCCGTCCGCCGGACCA |
| SEQ ID | MG29 O-tRNA | CCGGCGGTAGTTCAGAGGGCAGAACGGCGGACTCTAAATC |
| NO: 26 | DNA sequence | CGCAGGTCAGGGGTTCAAATCCCCTCCGCCGGACCA |
| SEQ ID | MG35 O-tRNA | CCGGCGGTAGTTCAGCAGGGCAGAACGGCGGACTCTAAAT |
| NO: 27 | DNA sequence | CCGCATGGCAGGGGTTCAAATCCCCTCCGCCGGACCA |
| SEQ ID | MG37 O-tRNA | CCGGCGGTAGTTCAGAGGGCAGAACGGCGGACTCTAAATC |
| NO: 28 | DNA sequence | CGCAGGTCACGGGTTCAAATCCCGTCCGCCGGACCA |
| SEQ ID | MG62 O-tRNA | CCGGAGGTAGTTCAGCAGGGCAGAACGGCGGACTCTAAAT |
| NO: 29 | DNA sequence | CCGCATGGCGCCGGTTCAAATCCGGCCCTTCGGACCA |
| SEQ ID | MG97 O-tRNA | CCGGTGTTAGTTCAGAGGGCAGAACGGCGGACTCTAAATCC |
| NO: 30 | DNA sequence | GCATGTCACGGGTTCAAATCCCGTACACCGGACCA |
| SEQ ID | MG109 O- | CCGACCTTAGTTCAGAGGGCAGAACGGCGGACTCTAAATCC |
| NO: 31 | tRNA DNA | GCATGTCAGGGGTTCAAATCCCCTAGGTCGGACCA |
| | sequence | |
| SEQ ID | Wild type Mj | CCGGCGGUAGUUCAGCCUGGUAGAACGGCGGACUGUAGA |
| NO: 32 | tyrosy1-tRNA | UCCGCAUGUCGCUGGUUCAAAUCCGGCCCGCCGGACCA |
| SEQ ID | Mj tyrosyl- | CCGGCGGTAGTTCAGCCTGGTAGAACGGCGGACTGTAGATC |
| NO: 33 | tRNA Wild type | CGCATGTCGCTGGTTCAAATCCGGCCCGCCGGACCA |
| | DNA sequence | |
| SEQ ID | Library tRNA | CNNGCNNTAGTTCAGCAGGGCAGAACGGCGGACTCTAAAT |
| NO: 34 | | CCGCATGGCWNRGGTTCAAATCCYNWNNGCNNGACCA |
| SEQ ID | Mj tyrosyl- | MDEFEMIKRNTSEIISEEELREVLKKDEKSAVIGFEPSGKIHLGH |
| NO: 35 | tRNA | YLQIKKMIDLQNAGFDIIIYLADLHAYLNQKGELDEIRKIGDYN |
| | synthetase “E9 | KKVFEAMGLKAKYVYGSEHGLDKDYTLNVYRLALKTTLKRA |
| | (D286R)” | RRSMELIAREDENPKVAEVIYPIMQVNGIHYEGVDVAVGGME |
| | amino acid | QRKIHMLARELLPKKVVCIHNPVLTGLDGEGKMSSSKGNFIAV |
| | sequence | DDSPEEIRAKIKKAYCPAGVVEGNPIMEIAKYFLEYPLTIKRPE |
| | | KFGGDLTVNSYEELESLFKNKELHPMRLKNAVAEELIKILEPIR |
| | | KRL |
| SEQ ID | O-tRNA | CCX₁X₂X₃X₄X₅UAGUUCAGX₆AGGGCAGAACGGCGGACUCUA |
| NO: 36 | consensus | AAUCCGCAX₇GX₈CX₉X₁₀X₁₁X₁₂GUCAAAUCX₁₃X₁₄X₁₅X₁₆X₁₇X₁₈ |
| | sequence | X₁₉X₂₀X₂₁GGACCA; |
| | | wherein: |
| | | X₁ is C or G; |
| | | X₂ is A or G; |
| | | X₃ is A, U or C; |
| | | X₄ is C or G; |
| | | X₅ is U or G; |
| | | X₆ is C or del; |
| | | X₇ is G or U; |
| | | X₈ is G or U; |
| | | X₉ is A or G; |
| | | X₁₀ is G or C; |
| | | X₁₁ is G, C, A or U; |
| | | X₁₂ is G or A; |
| | | X₁₃ is C or U; |
| | | X₁₄ is G, C, A or U; |
| | | X₁₅ is G or C; |
| | | X₁₆ is U or C; |
| | | X₁₇ is A or C; |
| | | X₁₈ is G or C; |
| | | X₁₉ is A, G or U; |
| | | X₂₀ is U or C; |
| | | X₂₁ is C or G. |
| SEQ ID | F13 | CCGGCGGUAGUUCAGCAGGGCAGAACGGCGGACUCUAAA |
| NO: 37 | | UCCGCAUGGCGCUGGUUCAAAUCCAGCCCGCCGGACCA |
| SEQ ID | F14 | CCGGCGGUAGUUCAGCAGGGCAGAACGGCGGACUCUAAA |
| NO: 38 | | UCCGCAUGGCGCAGGUUCAAAUCCUGCCCGCCGGACCA |
| SEQ ID | Mj tyrosyl- | MDEFEMIKRNTSEIVSEEELREVLKKDEKSAVIGFEPSGKIHLG |
| NO: 39 | tRNA | HYLQIKKMIDLQNAGFDIIIYLADLHAYLNQKGELDEIRKIGDY |
| | synthetase “E9 | NKKVFEAMGLKAKYVYGSEHGLDKDYTLNVYRLALKTTLKR |
| | (E9VR)” | ARRSMELIAREDENPKVAEVIYPIMQVNGIHYEGVDVAVGGM |
| | amino acid | EQRKIHMLARELLPKKVVCIHNPVLTGLDGEGKMSSSKGNFIA |
| | sequence; has | VDDSPEEIRAKIKKAYCPAGVVEGNPIMEIAKYFLEYPLTIKRP |
| | I15V and | EKFGGDLTVNSYEELESLFKNKELHPMRLKNAVAEELIKILEPI |
| | D286R | RKRL |
| | mutations | |
| SEQ ID | E9_I15V_D286R_ | MDEFEMIKRNASEIVSEEELREVLKKDEKSAVIGFEPSGKIHLG |
| NO: 40 | T11A | HYLQIKKMIDLQNAGFDIIIYLADLHAYLNQKGELDEIRKIGDY |
| | | NKKVFEAMGLKAKYVYGSEHGLDKDYTLNVYRLALKTTLKR |
| | | ARRSMELIAREDENPKVAEVIYPIMQVNGIHYEGVDVAVGGM |
| | | EQRKIHMLARELLPKKVVCIHNPVLTGLDGEGKMSSSKGNFIA |
| | | VDDSPEEIRAKIKKAYCPAGVVEGNPIMEIAKYFLEYPLTIKRP |
| | | EKFGGDLTVNSYEELESLFKNKELHPMRLKNAVAEELIKILEPI |
| | | RKRL |
| SEQ ID | E9_I15V_D286R_ | MDEFEMIKRNTSEIVSEEELREVLKKGEKSAVIGFEPSGKIHLG |
| NO: 41 | D27G | HYLQIKKMIDLQNAGFDIIIYLADLHAYLNOKGELDEIRKIGDY |
| | | NKKVFEAMGLKAKYVYGSEHGLDKDYTLNVYRLALKTTLKR |
| | | ARRSMELIAREDENPKVAEVIYPIMQVNGIHYEGVDVAVGGM |
| | | EQRKIHMLARELLPKKVVCIHNPVLTGLDGEGKMSSSKGNFIA |
| | | VDDSPEEIRAKIKKAYCPAGVVEGNPIMEIAKYFLEYPLTIKRP |
| | | EKFGGDLTVNSYEELESLFKNKELHPMRLKNAVAEELIKILEPI |
| | | RKRL |
| SEQ ID | E9_I15V_D286R_ | MDEFEMIKRNTSEIVSEEELREVLKKDEKSAVIGFEPSGKIHLG |
| NO: 42 | M96I | HYLQIKKMIDLQNAGFDIIIYLADLHAYLNQKGELDEIRKIGDY |
| | | NKKVFEAIGLKAKYVYGSEHGLDKDYTLNVYRLALKTTLKR |
| | | ARRSMELIAREDENPKVAEVIYPIMQVNGIHYEGVDVAVGGM |
| | | EQRKIHMLARELLPKKVVCIHNPVLTGLDGEGKMSSSKGNFIA |
| | | VDDSPEEIRAKIKKAYCPAGVVEGNPIMEIAKYFLEYPLTIKRP |
| | | EKFGGDLTVNSYEELESLFKNKELHPMRLKNAVAEELIKILEPI |
| | | RKRL |
| SEQ ID | E9_I15V_D286R_ | MDEFEMIKRNTSEIVSEEELREVLKKDEKSAVIGFEPSGKIHLG |
| NO: 43 | G97D | HYLQIKKMIDLQNAGFDIIIYLADLHAYLNQKGELDEIRKIGDY |
| | | NKKVFEAMDLKAKYVYGSEHGLDKDYTLNVYRLALKTTLKR |
| | | ARRSMELIAREDENPKVAEVIYPIMQVNGIHYEGVDVAVGGM |
| | | EQRKIHMLARELLPKKVVCIHNPVLTGLDGEGKMSSSKGNFIA |
| | | VDDSPEEIRAKIKKAYCPAGVVEGNPIMEIAKYFLEYPLTIKRP |
| | | EKFGGDLTVNSYEELESLFKNKELHPMRLKNAVAEELIKILEPI |
| | | RKRL |
| SEQ ID | E9_I15V_D286R_ | MDEFEMIKRNTSEIVSEEELREVLKKDEKSAVIGFEPSGKIHLG |
| NO: 44 | K101R | HYLQIKKMIDLQNAGFDIIIYLADLHAYLNQKGELDEIRKIGDY |
| | | NKKVFEAMGLKARYVYGSEHGLDKDYTLNVYRLALKTTLKR |
| | | ARRSMELIAREDENPKVAEVIYPIMQVNGIHYEGVDVAVGGM |
| | | EQRKIHMLARELLPKKVVCIHNPVLTGLDGEGKMSSSKGNFIA |
| | | VDDSPEEIRAKIKKAYCPAGVVEGNPIMEIAKYFLEYPLTIKRP |
| | | EKFGGDLTVNSYEELESLFKNKELHPMRLKNAVAEELIKILEPI |
| | | RKRL |
| SEQ ID | Mj tyrosyl- | MDEFEMIKRNTSEIISEEELREVLKKDEKSAVIGFEPSGKIHLGH |
| NO: 45 | tRNA (E9) | YLQIKKMIDLQNAGFDIIIYLADLHAYLNQKGELDEIRKIGDYN |
| | synthetase | KKVFEAMGLKAKYVYGSEHGLDKDYTLNVYRLALKTTLKRA |
| | amino acid | RRSMELIAREDENPKVAEVIYPIMQVNGIHYEGVDVAVGGME |
| | sequence | QRKIHMLARELLPKKVVCIHNPVLTGLDGEGKMSSSKGNFIAV |
| | | DDSPEEIRAKIKKAYCPAGVVEGNPIMEIAKYFLEYPLTIKRPE |
| | | KFGGDLTVNSYEELESLFKNKELHPMDLKNAVAEELIKILEPIR |
| | | KRL |
| SEQ ID | E9_I15V_D286R_ | MDEFEMIKRNASEIVSEEELREVLKKGEKSAVIGFEPSGKIHLG |
| NO: 46 | T11A_D27G_ | HYLQIKKMIDLQNAGFDIIIYLADLHAYLNQKGELDEIRKIGDY |
| | M96I_G97D_ | NKKVFEAIDLKARYVYGSEHGLDKDYTLNVYRLALKTTLKRA |
| | K101R | RRSMELIAREDENPKVAEVIYPIMQVNGIHYEGVDVAVGGME |
| | | QRKIHMLARELLPKKVVCIHNPVLTGLDGEGKMSSSKGNFIAV |
| | | DDSPEEIRAKIKKAYCPAGVVEGNPIMEIAKYFLEYPLTIKRPE |
| | | KFGGDLTVNSYEELESLFKNKELHPMRLKNAVAEELIKILEPIR |
| | | KRL |
| SEQ ID | E9_I15V_D286R_ | MDEFEMIKRNASEIVSEEELREVLKKDEKSAVIGFEPSGKIHLG |
| NO: 47 | T11A_M96I_ | HYLQIKKMIDLQNAGFDIIIYLADLHAYLNQKGELDEIRKIGDY |
| | K101R | NKKVFEAIGLKARYVYGSEHGLDKDYTLNVYRLALKTTLKRA |
| | | RRSMELIAREDENPKVAEVIYPIMQVNGIHYEGVDVAVGGME |
| | | QRKIHMLARELLPKKVVCIHNPVLTGLDGEGKMSSSKGNFIAV |
| | | DDSPEEIRAKIKKAYCPAGVVEGNPIMEIAKYFLEYPLTIKRPE |
| | | KFGGDLTVNSYEELESLFKNKELHPMRLKNAVAEELIKILEPIR |
| | | KRL |
| SEQ ID | E9_I15V_D286R_ | MDEFEMIKRNASEIVSEEELREVLKKGEKSAVIGFEPSGKIHLG |
| NO: 48 | T11A_D27G_ | HYLQIKKMIDLQNAGFDIIIYLADLHAYLNQKGELDEIRKIGDY |
| | M96I_G97D | NKKVFEAIDLKAKYVYGSEHGLDKDYTLNVYRLALKTTLKR |
| | | ARRSMELIAREDENPKVAEVIYPIMQVNGIHYEGVDVAVGGM |
| | | EQRKIHMLARELLPKKVVCIHNPVLTGLDGEGKMSSSKGNFIA |
| | | VDDSPEEIRAKIKKAYCPAGVVEGNPIMEIAKYFLEYPLTIKRP |
| | | EKFGGDLTVNSYEELESLFKNKELHPMRLKNAVAEELIKILEPI |
| | | RKRL |
| SEQ ID | E9_115V_D286R_ | MDEFEMIKRNASEIVSEEELREVLKKDEKSAVIGFEPSGKIHLG |
| NO: 49 | T11A_G97D_ | HYLQIKKMIDLQNAGFDIIIYLADLHAYLNQKGELDEIRKIGDY |
| | K101R | NKKVFEAMDLKARYVYGSEHGLDKDYTLNVYRLALKTTLKR |
| | | ARRSMELIAREDENPKVAEVIYPIMQVNGIHYEGVDVAVGGM |
| | | EQRKIHMLARELLPKKVVCIHNPVLTGLDGEGKMSSSKGNFIA |
| | | VDDSPEEIRAKIKKAYCPAGVVEGNPIMEIAKYFLEYPLTIKRP |
| | | EKFGGDLTVNSYEELESLFKNKELHPMRLKNAVAEELIKILEPI |
| | | RKRL |
| SEQ ID | E9_I15V_D286R_ | MDEFEMIKRNASEIVSEEELREVLKKGEKSAVIGFEPSGKIHLG |
| NO: 50 | T11A_D27G_ | HYLQIKKMIDLQNAGFDIIIYLADLHAYLNQKGELDEIRKIGDY |
| | M96I | NKKVFEAIGLKAKYVYGSEHGLDKDYTLNVYRLALKTTLKR |
| | | ARRSMELIAREDENPKVAEVIYPIMQVNGIHYEGVDVAVGGM |
| | | EQRKIHMLARELLPKKVVCIHNPVLTGLDGEGKMSSSKGNFIA |
| | | VDDSPEEIRAKIKKAYCPAGVVEGNPIMEIAKYFLEYPLTIKRP |
| | | EKFGGDLTVNSYEELESLFKNKELHPMRLKNAVAEELIKILEPI |
| | | RKRL |
| SEQ ID | E9VR_5mut_ | MDEFEMIKRNASEIVSEEELREVLKKGEKSAVIGFEPSGKIHLG |
| NO: 51 | F261P_P284S | HYLQIKKMIDLQNAGFDIIIYLADLHAYLNQKGELDEIRKIGDY |
| | | NKKVFEAIDLKARYVYGSEHGLDKDYTLNVYRLALKTTLKRA |
| | | RRSMELIAREDENPKVAEVIYPIMQVNGIHYEGVDVAVGGME |
| | | QRKIHMLARELLPKKVVCIHNPVLTGLDGEGKMSSSKGNFIAV |
| | | DDSPEEIRAKIKKAYCPAGVVEGNPIMEIAKYFLEYPLTIKRPE |
| | | KPGGDLTVNSYEELESLFKNKELHSMRLKNAVAEELIKILEPIR |
| | | KRL |
| SEQ ID | E9VR_5mut_ | MDEFEMIKRNASEIVSEEELREVLKKGEKSAVIGFEPSGKIHLG |
| NO: 52 | R257W_ F261P | HYLQIKKMIDLQNAGFDIIIYLADLHAYLNOKGELDEIRKIGDY |
| | | NKKVFEAIDLKARYVYGSEHGLDKDYTLNVYRLALKTTLKRA |
| | | RRSMELIAREDENPKVAEVIYPIMQVNGIHYEGVDVAVGGME |
| | | QRKIHMLARELLPKKVVCIHNPVLTGLDGEGKMSSSKGNFIAV |
| | | DDSPEEIRAKIKKAYCPAGVVEGNPIMEIAKYFLEYPLTIKWPE |
| | | KPGGDLTVNSYEELESLFKNKELHPMRLKNAVAEELIKILEPIR |
| | | KRL |
| SEQ ID | E9VR_5mut_ | MDEFEMIKRNASEIVSEEELREVLKKGEKSAVIGFEPSGKIHLG |
| NO: 53 | R257W_ | HYLQIKKMIDLQNAGFDIIIYLADLHAYLNOKGELDEIRKIGDY |
| | F261P_P284S | NKKVFEAIDLKARYVYGSEHGLDKDYTLNVYRLALKTTLKRA |
| | | RRSMELIAREDENPKVAEVIYPIMQVNGIHYEGVDVAVGGME |
| | | QRKIHMLARELLPKKVVCIHNPVLTGLDGEGKMSSSKGNFIAV |
| | | DDSPEEIRAKIKKAYCPAGVVEGNPIMEIAKYFLEYPLTIKWPE |
| | | KPGGDLTVNSYEELESLFKNKELHSMRLKNAVAEELIKILEPIR |
| | | KRL |
| SEQ ID | E9VR_5mut_ | MDEFEMIKRNASEIVSEEELREVLKKGEKSAVIGFEPSGKIHLG |
| NO: 54 | R257W_ | HYLQIKKMIDLQNAGFDIIIYLADLHAYLNQKGELDEIRKIGDY |
| | F261P_R286Y | NKKVFEAIDLKARYVYGSEHGLDKDYTLNVYRLALKTTLKRA |
| | | RRSMELIAREDENPKVAEVIYPIMQVNGIHYEGVDVAVGGME |
| | | QRKIHMLARELLPKKVVCIHNPVLTGLDGEGKMSSSKGNFIAV |
| | | DDSPEEIRAKIKKAYCPAGVVEGNPIMEIAKYFLEYPLTIKWPE |
| | | KPGGDLTVNSYEELESLFKNKELHPMYLKNAVAEELIKILEPIR |
| | | KRL |
| SEQ ID | E9VR_5mut_ | MDEFEMIKRNASEIVSEEELREVLKKGEKSAVIGFEPSGKIHLG |
| NO: 55 | R286Y | HYLQIKKMIDLQNAGFDIIIYLADLHAYLNQKGELDEIRKIGDY |
| | | NKKVFEAIDLKARYVYGSEHGLDKDYTLNVYRLALKTTLKRA |
| | | RRSMELIAREDENPKVAEVIYPIMQVNGIHYEGVDVAVGGME |
| | | QRKIHMLARELLPKKVVCIHNPVLTGLDGEGKMSSSKGNFIAV |
| | | DDSPEEIRAKIKKAYCPAGVVEGNPIMEIAKYFLEYPLTIKRPE |
| | | KFGGDLTVNSYEELESLFKNKELHPMYLKNAVAEELIKILEPIR |
| | | KRL |
| SEQ ID | E9VR_5mut_ | MDEFEMIKRNASEIVSEEELREVLKKGEKSAVIGFEPSGKIHLG |
| NO: 56 | F261P_R286Y | HYLQIKKMIDLQNAGFDIIIYLADLHAYLNQKGELDEIRKIGDY |
| | | NKKVFEAIDLKARYVYGSEHGLDKDYTLNVYRLALKTTLKRA |
| | | RRSMELIAREDENPKVAEVIYPIMQVNGIHYEGVDVAVGGME |
| | | QRKIHMLARELLPKKVVCIHNPVLTGLDGEGKMSSSKGNFIAV |
| | | DDSPEEIRAKIKKAYCPAGVVEGNPIMEIAKYFLEYPLTIKRPE |
| | | KPGGDLTVNSYEELESLFKNKELHPMYLKNAVAEELIKILEPIR |
| | | KRL |
| SEQ ID | DNA encoding | ATGGATGAGTTTGAGATGATTAAACGTAATACGAGTGAAAT |
| NO: 57 | E9VR (has | TGTCAGTGAGGAAGAATTGCGCGAGGTCCTTAAAAAAGAT |
| | I15V and | GAAAAATCGGCCGTTATCGGCTTCGAGCCAAGTGGTAAAAT |
| | D286R | CCATTTAGGGCACTACCTGCAGATCAAAAAGATGATTGATT |
| | mutations) | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TGGGTCTTAAAGCGAAATATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGTTTGGCGGGGATTTAACAGTTAACAGCTATGAAGA |
| | | ACTTGAAAGTCTTTTCAAAAATAAGGAGTTGCATCCGATGC |
| | | GCTTAAAGAACGCTGTCGCGGAGGAATTGATTAAAATCCTG |
| | | GAGCCTATTCGTAAACGTCTGTAA |
| SEQ ID | DNA encoding | ATGGATGAGTTTGAGATGATTAAACGTAATGCGAGTGAAAT |
| NO: 58 | E9VR_T11A_ | TGTCAGTGAGGAAGAATTGCGCGAGGTCCTTAAAAAAGGC |
| | D27G_M96I_ | GAAAAATCGGCCGTTATCGGCTTCGAGCCAAGTGGTAAAAT |
| | G97D_K101R | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TTGATCTTAAAGCGCGTTATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGTTTGGCGGGGATTTAACAGTTAACAGCTATGAAGA |
| | | ACTTGAAAGTCTTTTCAAAAATAAGGAGTTGCATCCGATGC |
| | | GCTTAAAGAACGCTGTCGCGGAGGAATTGATTAAAATCCTG |
| | | GAGCCTATTCGTAAACGTCTGTAA |
| SEQ ID | DNA encoding | ATGGATGAGTTTGAGATGATTAAACGTAATGCGAGTGAAAT |
| NO: 59 | I15V_D286R_ | TGTCAGTGAGGAAGAATTGCGCGAGGTCCTTAAAAAAGAT |
| | T11A | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TGGGTCTTAAAGCGAAATATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGTTTGGCGGGGATTTAACAGTTAACAGCTATGAAGA |
| | | ACTTGAAAGTCTTTTCAAAAATAAGGAGTTGCATCCGATGC |
| | | GCTTAAAGAACGCTGTCGCGGAGGAATTGATTAAAATCCTG |
| | | GAGCCTATTCGTAAACGTCTGTAA |
| SEQ ID | DNA encoding | ATGGATGAGTTTGAGATGATTAAACGTAATACGAGTGAAAT |
| NO: 60 | I15V_D286R_ | TGTCAGTGAGGAAGAATTGCGCGAGGTCCTTAAAAAAGGC |
| | D27G | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TGGGTCTTAAAGCGAAATATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGTTTGGCGGGGATTTAACAGTTAACAGCTATGAAGA |
| | | ACTTGAAAGTCTTTTCAAAAATAAGGAGTTGCATCCGATGC |
| | | GCTTAAAGAACGCTGTCGCGGAGGAATTGATTAAAATCCTG |
| | | GAGCCTATTCGTAAACGTCTGTAA |
| SEQ ID | DNA encoding | ATGGATGAGTTTGAGATGATTAAACGTAATACGAGTGAAAT |
| NO: 61 | I15V_D286R_ | TGTCAGTGAGGAAGAATTGCGCGAGGTCCTTAAAAAAGAT |
| | M96I | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TTGGTCTTAAAGCGAAATATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGTTTGGCGGGGATTTAACAGTTAACAGCTATGAAGA |
| | | ACTTGAAAGTCTTTTCAAAAATAAGGAGTTGCATCCGATGC |
| | | GCTTAAAGAACGCTGTCGCGGAGGAATTGATTAAAATCCTG |
| | | GAGCCTATTCGTAAACGTCTGTAA |
| SEQ ID | DNA encoding | ATGGATGAGTTTGAGATGATTAAACGTAATACGAGTGAAAT |
| NO: 62 | I15V_D286R_ | TGTCAGTGAGGAAGAATTGCGCGAGGTCCTTAAAAAAGAT |
| | G97D | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TGGATCTTAAAGCGAAATATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGTTTGGCGGGGATTTAACAGTTAACAGCTATGAAGA |
| | | ACTTGAAAGTCTTTTCAAAAATAAGGAGTTGCATCCGATGC |
| | | GCTTAAAGAACGCTGTCGCGGAGGAATTGATTAAAATCCTG |
| | | GAGCCTATTCGTAAACGTCTGTAA |
| SEQ ID | DNA encoding | ATGGATGAGTTTGAGATGATTAAACGTAATACGAGTGAAAT |
| NO: 63 | I15V_D286R_ | TGTCAGTGAGGAAGAATTGCGCGAGGTCCTTAAAAAAGAT |
| | K101R | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TGGGTCTTAAAGCGCGTTATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGTTTGGCGGGGATTTAACAGTTAACAGCTATGAAGA |
| | | ACTTGAAAGTCTTTTCAAAAATAAGGAGTTGCATCCGATGC |
| | | GCTTAAAGAACGCTGTCGCGGAGGAATTGATTAAAATCCTG |
| | | GAGCCTATTCGTAAACGTCTGTAA |
| SEQ ID | DNA encoding | ATGGATGAGTTTGAGATGATTAAACGTAATGCGAGTGAAAT |
| NO: 64 | E9VR_T11A_ | TGTCAGTGAGGAAGAATTGCGCGAGGTCCTTAAAAAAGAT |
| | M96I_K101R | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TTGGTCTTAAAGCGCGTTATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGTTTGGCGGGGATTTAACAGTTAACAGCTATGAAGA |
| | | ACTTGAAAGTCTTTTCAAAAATAAGGAGTTGCATCCGATGC |
| | | GCTTAAAGAACGCTGTCGCGGAGGAATTGATTAAAATCCTG |
| | | GAGCCTATTCGTAAACGTCTGTAA |
| SEQ ID | DNA encoding | ATGGATGAGTTTGAGATGATTAAACGTAATGCGAGTGAAAT |
| NO: 65 | E9VR_T11A_ | TGTCAGTGAGGAAGAATTGCGCGAGGTCCTTAAAAAAGGC |
| | D27G_M96I_ | GAAAAATCGGCCGTTATCGGCTTCGAGCCAAGTGGTAAAAT |
| | G97D | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TTGATCTTAAAGCGAAATATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGTTTGGCGGGGATTTAACAGTTAACAGCTATGAAGA |
| | | ACTTGAAAGTCTTTTCAAAAATAAGGAGTTGCATCCGATGC |
| | | GCTTAAAGAACGCTGTCGCGGAGGAATTGATTAAAATCCTG |
| | | GAGCCTATTCGTAAACGTCTGTAA |
| SEQ ID | DNA encoding | ATGGATGAGTTTGAGATGATTAAACGTAATGCGAGTGAAAT |
| NO: 66 | E9VR_T11A_ | TGTCAGTGAGGAAGAATTGCGCGAGGTCCTTAAAAAAGAT |
| | G97D_K101R | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TGGATCTTAAAGCGCGTTATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGTTTGGCGGGGATTTAACAGTTAACAGCTATGAAGA |
| | | ACTTGAAAGTCTTTTCAAAAATAAGGAGTTGCATCCGATGC |
| | | GCTTAAAGAACGCTGTCGCGGAGGAATTGATTAAAATCCTG |
| | | GAGCCTATTCGTAAACGTCTGTAA |
| SEQ ID | DNA encoding | ATGGATGAGTTTGAGATGATTAAACGTAATGCGAGTGAAAT |
| NO: 67 | E9VR_T11A_ | TGTCAGTGAGGAAGAATTGCGCGAGGTCCTTAAAAAAGGC |
| | D27G_M96I | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TTGGTCTTAAAGCGAAATATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGTTTGGCGGGGATTTAACAGTTAACAGCTATGAAGA |
| | | ACTTGAAAGTCTTTTCAAAAATAAGGAGTTGCATCCGATGC |
| | | GCTTAAAGAACGCTGTCGCGGAGGAATTGATTAAAATCCTG |
| | | GAGCCTATTCGTAAACGTCTGTAA |
| SEQ ID | E9VR_5mut_ | ATGGATGAGTTTGAGATGATTAAACGTAATGCGAGTGAAAT |
| NO: 68 | F261P_P284S | TGTCAGTGAGGAAGAATTGCGCGAGGTCCTTAAAAAAGGC |
| | | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TTGATCTTAAAGCGCGTTATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGCCGGGGGGGGATTTAACAGTTAACAGCTATGAAGA |
| | | ACTTGAAAGTCTTTTCAAAAATAAGGAGTTGCATAGCATGC |
| | | GCTTAAAGAACGCTGTCGCGGAGGAATTGATTAAAATCCTG |
| | | GAGCCTATTCGTAAACGTCTGTAA |
| SEQ ID | E9VR_5mut_ | ATGGATGAGTTTGAGATGATTAAACGTAATGCGAGTGAAAT |
| NO: 69 | R257W_F261P | TGTCAGTGAGGAAGAATTGCGCGAGGTCCTTAAAAAAGGC |
| | | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TTGATCTTAAAGCGCGTTATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAATGGCCCG |
| | | AGAAGCCGGGCGGGGATTTAACAGTTAACAGCTATGAAGA |
| | | ACTTGAAAGTCTTTTCAAAAATAAGGAGTTGCATCCGATGC |
| | | GCTTAAAGAACGCTGTCGCGGAGGAATTGATTAAAATCCTG |
| | | GAGCCTATTCGTAAACGTCTGTAA |
| SEQ ID | E9VR_5mut_ | ATGGATGAGTTTGAGATGATTAAACGTAATGCGAGTGAAAT |
| NO: 70 | R257W_ | TGTCAGTGAGGAAGAATTGCGCGAGGTCCTTAAAAAAGGC |
| | F261P_P284S | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TTGATCTTAAAGCGCGTTATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAATGGCCCG |
| | | AGAAGCCGGGCGGGGATTTAACAGTTAACAGCTATGAAGA |
| | | ACTTGAAAGTCTTTTCAAAAATAAGGAGTTGCATAGCATGC |
| | | GCTTAAAGAACGCTGTCGCGGAGGAATTGATTAAAATCCTG |
| | | GAGCCTATTCGTAAACGTCTGTAA |
| SEQ ID | E9VR_5mut_ | ATGGATGAGTTTGAGATGATTAAACGTAATGCGAGTGAAAT |
| NO: 71 | R257W_ | TGTCAGTGAGGAAGAATTGCGCGAGGTCCTTAAAAAAGGC |
| | F261P_R286Y | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TTGATCTTAAAGCGCGTTATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAATGGCCCG |
| | | AGAAGCCGGGCGGGGATTTAACAGTTAACAGCTATGAAGA |
| | | ACTTGAAAGTCTTTTCAAAAATAAGGAGTTGCATCCGATGT |
| | | ATTTAAAGAACGCTGTCGCGGAGGAATTGATTAAAATCCTG |
| | | GAGCCTATTCGTAAACGTCTGTAA |
| SEQ ID | E9VR_5mut_ | ATGGATGAGTTTGAGATGATTAAACGTAATGCGAGTGAAAT |
| NO: 72 | R286Y | TGTCAGTGAGGAAGAATTGCGCGAGGTCCTTAAAAAAGGC |
| | | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TTGATCTTAAAGCGCGTTATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGTTTGGCGGGGATTTAACAGTTAACAGCTATGAAGA |
| | | ACTTGAAAGTCTTTTCAAAAATAAGGAGTTGCATCCGATGT |
| | | ATTTAAAGAACGCTGTCGCGGAGGAATTGATTAAAATCCTG |
| | | GAGCCTATTCGTAAACGTCTGTAA |
| SEQ ID | E9VR_5mut_ | ATGGATGAGTTTGAGATGATTAAACGTAATGCGAGTGAAAT |
| NO: 73 | F261P_R286Y | TGTCAGTGAGGAAGAATTGCGCGAGGTCCTTAAAAAAGGC |
| | | GAAAAATCGGCCGTTATCGGCTTCGAGCCAAGTGGTAAAAT |
| | | CCATTTAGGGCACTACCTGCAGATCAAAAAGATGATTGATT |
| | | TACAGAACGCGGGCTTTGACATCATTATCTACCTTGCTGAC |
| | | TTGCATGCGTATTTGAACCAAAAGGGCGAGCTTGATGAAAT |
| | | CCGCAAAATTGGAGATTACAATAAAAAAGTTTTCGAAGCCA |
| | | TTGATCTTAAAGCGCGTTATGTCTACGGGTCTGAACACGGT |
| | | TTGGATAAGGATTACACTTTGAACGTATATCGCTTGGCGCT |
| | | TAAAACCACTTTGAAACGTGCTCGCCGCTCTATGGAATTAA |
| | | TTGCACGCGAGGACGAAAACCCTAAAGTCGCGGAGGTCAT |
| | | CTACCCTATCATGCAGGTAAACGGTATCCATTATGAGGGGG |
| | | TTGATGTAGCTGTTGGTGGTATGGAGCAACGTAAAATTCAC |
| | | ATGCTGGCACGCGAGCTTCTGCCGAAAAAGGTTGTCTGTAT |
| | | TCACAACCCGGTTCTTACGGGGTTAGATGGAGAGGGAAAA |
| | | ATGAGCTCTAGCAAGGGCAATTTCATTGCGGTAGATGACTC |
| | | TCCGGAAGAAATTCGTGCAAAAATCAAGAAGGCGTACTGT |
| | | CCGGCCGGCGTAGTCGAAGGAAATCCGATTATGGAGATTGC |
| | | AAAGTACTTTCTGGAGTATCCATTGACGATCAAACGCCCCG |
| | | AGAAGCCGGGCGGGGATTTAACAGTTAACAGCTATGAAGA |
| | | ACTTGAAAGTCTTTTCAAAAATAAGGAGTTGCATCCGATGT |
| | | ATTTAAAGAACGCTGTCGCGGAGGAATTGATTAAAATCCTG |
| | | GAGCCTATTCGTAAACGTCTGTAA |

1. An orthogonal tRNA synthetase (O-RS) comprising a substitution of at least one of the following residues as compared to a wild-type M. janneschii tRNA synthetase (SEQ ID NO:45):
(a) T11,
(b) I15,
(c) D27,
(d) M96,
(e) G97, and
(f) K101.

2. The O-RS of claim 1, wherein the orthogonal tRNA synthetase comprises at least 85% sequence identity to SEQ ID NO: 45 but is not identical to SEQ ID NO: 35.

3. The O-RS of claim 1, wherein the O-RS comprises at least one of the following substitutions:
(a) T11A, T11V, T11I, T11L, or T11G;
(b) I15V, I15A, I15L, or I15G;
(c) D27G, D27A, D27V, D27I, or D27L;
(d) M96I, M96A, M96V, M96L, or M96G;
(e) G97D or G97E; and
(f) K101R, or K101H.

4. The O-RS of claim 3, wherein the O-RS comprises at least one of the following substitutions:
(a) T11A,
(b) I15V,
(c) D27G,
(d) M96I,
(e) G97D, and
(f) K101R.

5. The O-RS claim 1, wherein the O-RS comprises an additional substitution of at least one of the following residues as compared to a wild-type M. janneschii tRNA synthetase (SEQ ID NO:45):
(a) R257,
(b) F261,
(c) P284,
(d) M285,
(e) D286, and
(f) G158.

6. The O-RS of claim 5, wherein the O-RS comprises at least one of the following substitutions:
(a) R257W, R257F, R257Y, or R257H,
(b) F261P,
(c) P284S, P284A, P284G, P284C, or P284V,
(d) M285D, or M285E,
(e) D286Y, D286W, D286F, D286H, or D286R, and
(f) G158D, or G158E.

7. The O-RS of claim 6, wherein the O-RS comprises at least one of the following substitutions:
(a) R257W,
(b) F261P,
(c) P284S,
(d) M285D,
(e) D286Y, and
(f) G158D.

8. The O-RS of claim 1, wherein the O-RS comprises a substitution at residue D286.

9. The O-RS of claim 1, wherein the O-RS comprises substitutions at residues 115 and D286.

10. The O-RS of claim 8, wherein the substitution at D286 is a D286F, D286W, D286H, D286K, D286V, D286Y, or a D286R substitution.

11. The O-RS of claim 10, wherein the substitution at D286 is D286Y.

12. The O-RS of claim 1, wherein the O-RS comprises at least one of the substitutions I15V and D286R.

13. The O-RS of claim 12, wherein the O-RS comprises the substitutions I15V and D286R.

14. The O-RS of claim 1, wherein the O-RS comprises an amino acid sequence that is at least 85% identical to a sequence selected from SEQ ID NOs: 39-44 and 46-56.

15. The O-RS of claim 14, wherein the amino acid sequence is at least 90% identical, at least 91% identical, at least 92% identical, at least 93% identical, at least 94% identical, at least 95% identical, at least 96% identical, at least 97% identical, at least 98% identical, or at least 98.7% identical to a sequence selected from SEQ ID NOs: 39-44 and 46-56.

16. The O-RS of claim 15, wherein the amino acid sequence is selected from SEQ ID NOs: 39-44 and 46-56.

17. An orthogonal translation system (OTS) comprising the O-RS of claim 1 and an orthogonal tRNA (0-tRNA).

18.-73. (canceled)

74. A cell comprising the OTS of claim 17.

75.-84. (canceled)

85. A polypeptide comprising at least one nsAA, wherein the polypeptide is produced by the OTS of claim 17.

86.-87. (canceled)

88. A polynucleotide comprising a nucleic acid sequence encoding an O-RS of claim 1.

89.-92. (canceled)

93. A vector comprising the polynucleotide of claim 88.

94.-95. (canceled)

96. A cell comprising the polynucleotide of claim 88.

97. A kit comprising the polynucleotide of claim 88, and instructions for use.

98. A method of producing a polypeptide comprising at least one nsAA, comprising expressing in a cell an O-tRNA and an O-RS of claim 1.

99.-132. (canceled)

133. A method of producing a polypeptide comprising at least one non-standard amino acid (nsAA), comprising providing:
i) an O-tRNA, wherein the O-tRNA is capable of being aminoacylated with at least one non-standard amino acid (nsAA) by an O-RS of claim 1;
ii) the O-RS; wherein the O-RS aminoacylates the O-tRNA with the nsAA; and
iii) a polynucleotide encoding the polypeptide, wherein the polynucleotide comprises at least one selector codon; and wherein the O-tRNA recognizes the selector codon.

134.-158. (canceled)
